(19) United States
(12) Patent Application Publication
(10) Pub. No.: US 2010/0113545 A1
(43) Pub. Date: May 6, 2010

(54) GLYT1 TRANSPORTER INHIBITORS AND USES THEREOF IN TREATMENT OF NEUROLOGICAL AND NEUROPSYCHIATRIC DISORDERS

(76) Inventors: Steven Coulton, Essex (GB); Roderick Alan Porter, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/524,593

(22) PCT Filed: Jan. 30, 2008
(86) PCT No.: PCT/EP08/51077

§ 371 Date: Jul. 27, 2009

(30) Foreign Application Priority Data
Feb. 1, 2007 (GB) 0701984.7
Aug. 31, 2007 (GB) 0716990.7

(51) Int. Cl. A61K 31/4166; C07D 235/02; A61P 25/18

(52) U.S. Cl. 514/386; 548/300.7

(57) ABSTRACT

Compounds of formula (I) or a salt thereof are provided:

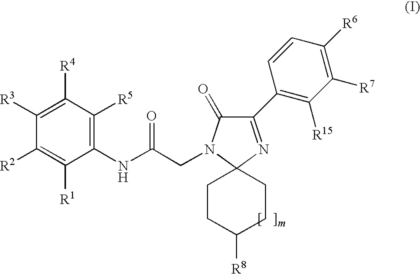

wherein R¹, R², R³, R⁴, R⁵, R⁶, R¹⁵, R⁷, R⁸ and m are as defined in the description. Uses of the compounds as medicaments, and in the manufacture of medicament for treating neurological and neuropsychiatric disorders, in particular psychoses, dementia or attention deficit disorder are also disclosed. The invention further comprises processes to make these compounds and pharmaceutical formulations thereof.

The present invention relates to compounds, processes for their preparation, pharmaceutical compositions and medicaments containing them and to their use in treating disorders mediated by GlyT1, including neurological and neuropsychiatric disorders, in particular psychoses, dementia or attention deficit disorder.

Molecular cloning has revealed the existence in mammalian brains of two classes of glycine transporters, termed GlyT1 and GlyT2. GlyT1 is found predominantly in the forebrain and its distribution corresponds to that of glutaminergic pathways and NMDA receptors (Smith, et al., Neuron, 8, 1992: 927-935). Molecular cloning has further revealed the existence of three variants of GlyT1, termed GlyT-1a, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995: 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science, 262, 689-695 (1993); Lipton and Rosenberg, New Engl. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Glycine transport inhibitors are already known in the art, for example as disclosed in published international patent application WO03/055478 (SmithKline Beecham).

However, there still remains the need to identify further compounds that can inhibit GlyT1 transporters, including those that inhibit GlyT1 transporters selectively over GlyT2 transporters.

It has now been found that a novel class of compounds inhibit GlyT1 transporters and are thus of potential utility in the treatment of certain neurological and neuropsychiatric disorders, including schizophrenia.

Thus, in the first aspect, there is provided a compound of formula (I) or a salt thereof:

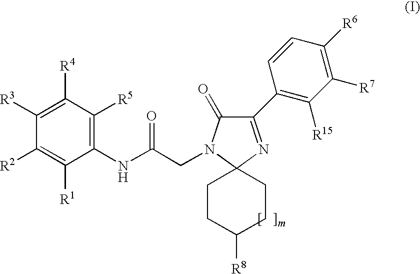

wherein:
R¹ is selected from hydrogen, C₁₋₄alkyl, C₁₋₄alkoxy, halo, haloC₁₋₄alkyl, haloC₁₋₄alkoxy, C₁₋₄alkylthio, C₃₋₆cycloalkyl, C₃₋₆cycloalkylC₁alkyl, C₁₋₄alkylsulfonyl, C₁₋₄alkoxyC₁₋₄alkyl, cyano and C(O)NR^{a}R^{b} (wherein R^{a} and R^{b} are independently selected from H and C₁₋₄alkyl, or R^{a} and R^{b}, together with the nitrogen atom to which they are attached, form a 4- to 7-membered ring);
R² is selected from hydrogen, C₁₋₄alkyl, C₁₋₄alkoxy, halo, haloC₁₋₄alkyl, haloC₁₋₄alkoxy, C₁₋₄alkylthio, C₃₋₆cycloalkyl, C₃₋₆cycloalkylC₁alkyl, C₁₋₄alkylsulfonyl, C₁₋₄alkoxyC₁₋₄alkyl, cyano and CONR^{c}R^{d} (wherein R^{c} and R^{d} are independently selected from H and C₁₋₄alkyl, or R^{c} and R^{d}, together with the nitrogen atom to which they are attached, form a 4- to 7-membered ring);
R³ is selected from hydrogen, C₁₋₄alkyl, C₁₋₄alkoxy, halo, haloC₁₋₄alkyl, haloC₁₋₄alkoxy, C₁₋₄alkylthio, C₃₋₆cycloalkyl, C₃₋₆cycloalkylC₁alkyl, C₁₋₄alkylsulfonyl, C₁₋₄alkoxyC₁₋₄alkyl, cyano and CONR^{e}R^{f} (wherein R^{e} and R^{f} are independently selected from H and C₁₋₄alkyl, or R^{e} and R^{f}, together with the nitrogen atom to which they are attached, form a 4- to 7-membered ring);
or R² and R³ together form a group selected from —O—CH₂—O— and —O—CH₂—CH₂—O—;
R⁴ is selected from hydrogen, C₁₋₄alkyl, C₁₋₄alkoxy, halo, haloC₁₋₄alkyl, haloC₁₋₄alkoxy, C₃₋₆cycloalkyl, C₃₋₆cycloalkylC₁₋₄alkyl, C₁₋₄alkylsulfonyl, C₁₋₄alkoxyC₁₋₄alkyl, cyano and CONR^{g}R^{h} (wherein R^{g} and R^{h} are independently selected from H and C₁₋₄alkyl, or R^{g} and R^{h}, together with the nitrogen atom to which they are attached, form a 4- to 7-membered ring);
R⁵ is selected from hydrogen, chloro, fluoro, C₁₋₄alkyl and CF₃;
R⁶ is selected from C₁₋₄alkoxyC₁₋₄alkyl, C₁₋₄alkylsulfonyl, COR⁹ (wherein R⁹ is hydrogen or C₁₋₄alkyl), CONRⁱR^{j} (wherein Rⁱ and R^{j} are independently selected from hydrogen and C₁₋₄alkyl or, together with the nitrogen atom to which they are attached, form a 4, 5 or 6-membered ring) and CHR^{k}NR^{l}R^{m} (wherein R^{k} is hydrogen or C₁₋₄alkyl and R^{l} and R^{m} are independently selected from hydrogen and C₁₋₄alkyl or R^{l} and R^{m}, together with the nitrogen atom to which they are attached, form a 4, 5 or 6-membered ring);
R¹⁵ is hydrogen or fluoro;
R⁷ is selected from hydrogen, C₁₋₄alkyl, C₁₋₄alkoxy, haloC₁₋₄alkoxy, halo, cyano, C₁₋₄alkoxyC₁₋₄alkoxy and C₁₋₄alkoxyC₁₋₄alkyl;
R⁸ is selected from hydrogen and methyl; and
m is selected from 0, 1 and 2.

The notations “C_{x-y}” and “Cₓ-C_{y}” are interchangeable.

As used herein, the term “C₁₋₄alkyl” refers to a straight or branched alkyl group of 1-4 carbon atoms in all isomeric forms. Examples include methyl, ethyl, propyl, isopropyl, butyl, isobutyl, sec-butyl and tert-butyl.

As used herein, the term “C₁₋₄alkoxy” refers to the group —O—C₁₋₄alkyl wherein C₁₋₄alkyl is as defined above.

As used herein, the term “C₁₋₄alkoxyC₁₋₄alkyl” refers to the group —(C₁₋₄alkyl)-O—(C₁₋₄alkyl), wherein C₁₋₄alkyl is as defined above.

As used herein, the term “C₁₋₄alkoxyC₁₋₄alkyoxy” refers to the group —OC₁₋₄alkyl-O—C₁₋₄alkyl, wherein C₁₋₄alkyl is as defined above.

As used herein, the term “C₃₋₆cycloalkyl” refers to a cycloalkyl group consisting of from 3 to 6 carbon atoms, ie cyclopropane, cyclobutane, cyclopentane or cyclohexane.

As used herein, the terms “halogen” and its abbreviation “halo” refer to fluorine, chlorine, bromine, or iodine.

As used herein, the term “haloC₁₋₄alkyl” refers to a C₁₋₄alkyl group as defined above which is substituted with any number of fluorine, chlorine, bromine, or iodine atoms, including with mixtures of those atoms. A haloC₁₋₄alkyl group may, for example contain 1, 2 or 3 halogen atoms. For example, a haloC₁₋₄alkyl group may have all hydrogen atoms replaced with halogen atoms. Examples of haloC₁₋₄alkyl groups include fluoromethyl, difluoromethyl and trifluoromethyl.

As used herein, the term “haloC₁₋₄alkoxy” refers to a C₁₋₄alkoxy group as defined above which is substituted with any number of fluorine, chlorine, bromine, or iodine atoms, including with mixtures of those atoms. A haloC₁₋₄alkoxy group may, for example contain 1, 2 or 3 halogen atoms. For example, a haloC₁₋₄alkoxy group may have all hydrogen atoms replaced with halogen atoms. Examples of haloC₁₋₄alkoxy groups include fluoromethyloxy, difluoromethyloxy and trifluoromethyloxy.

As used herein the term “cyano” refers to a group —CN.

As used herein, the term “C₁₋₄alkylsulfonyl” refers to a group —SO₂(C₁₋₄alkyl). An example is —SO₂CH₃.

As used herein, the term “C₁₋₄alkylthio” refers to a group —S—(C₁₋₄alkyl). An example is —SCH₃.

R^{a} and R^{b}, together with the nitrogen atom to which they are attached, may form a saturated 4- to 7-membered ring, ie an azetidinyl, pyrrolidinyl, piperidyl, or azepanyl group. Similarly, R^{c} and R^{d}, R^{e} and R^{f}, R^{g} and R^{h}, Rⁱ and R^{j}, and R^{l} and R^{m} may form such a group within the definition of formula (I) above.

In one embodiment, R¹ is selected from hydrogen, C₁₋₂alkyl, C₁₋₂alkoxy, halo, haloC₁₋₂alkyl, haloC₁₋₂alkoxy, C₁₋₂alkylthio, C₁₋₂alkylsulfonyl, C₁₋₂alkoxyC₁₋₂alkyl and cyano.

In one embodiment, R¹ is hydrogen.

In one embodiment, R² is selected from hydrogen, C₁₋₂alkyl, C₁₋₂alkoxy, halo, haloC₁₋₂alkyl, haloC₁₋₂alkoxy, C₁₋₂alkylsulfonyl, C₁₋₂alkoxyC₁₋₂alkyl and cyano.

In one embodiment, R² is halo. In one embodiment, R² is fluoro.

In one embodiment, R² is haloC₁₋₄lkyl. In one embodiment, R² is haloC₁₋₂alkyl. In one embodiment, R² is CF₃.

In one embodiment, R³ is selected from hydrogen, C₁₋₂alkyl, C₁₋₂alkoxy, halo, haloC₁₋₂alkyl, haloC₁₋₂alkoxy, C₁₋₂alkylthio, C₁₋₂alkylsulfonyl, C₁₋₂alkoxyC₁₋₂alkyl and cyano. In a further embodiment, R³ is hydrogen.

In one embodiment, R⁴ is selected from hydrogen, C₁₋₂alkyl, C₁₋₂alkoxy, halo, haloC₁₋₂alkyl, haloC₁₋₂alkoxy, C₁₋₂alkylsulfonyl, C₁₋₂alkoxyC₁₋₂alkyl and cyano. In a further embodiment, R⁴ is halo. In a further embodiment, R⁴ is fluoro. In one embodiment, R⁴ is hydrogen.

In one embodiment, R⁵ is hydrogen.

In one embodiment, R¹ is hydrogen, R² is halo or haloC₁₋₄lkyl, R³ is hydrogen, R⁴ is halo or hydrogen, and R⁵ is hydrogen.

In one embodiment, R⁶ is selected from C₁₋₂alkoxyC₁₋₂alkyl, C₁₋₂alkylsulfonyl, COR⁹ (wherein R⁹ is hydrogen or C₁₋₂alkyl), CONRⁱR^{j} (wherein Rⁱ and R^{j} are independently selected from hydrogen and C₁₋₂alkyl or, together with the nitrogen atom to which they are attached, form a 4, 5 or 6-membered ring) and CHR^{k}NR^{l}R^{m} (wherein R^{k} is hydrogen or C₁₋₄alkyl and R^{l} and R^{m} are independently selected from hydrogen and C₁₋₂alkyl or R^{l} and R^{m}, together with the nitrogen atom to which they are attached, form a 4, 5 or 6-membered ring).

In one embodiment, R⁶ is selected from C₁₋₄alkoxyC₁₋₄alkyl, C₁₋₄alkylsulfonyl, COR⁹ (wherein R⁹ is hydrogen or C₁₋₄alkyl), CONRⁱR^{j} (wherein Rⁱ and R^{j} are independently selected from hydrogen and C₁₋₄alkyl) and CHR^{k}NR^{l}R^{m} (wherein R^{k} is hydrogen or C₁₋₄alkyl and R^{l} and R^{m} are independently selected from hydrogen and C₁₋₄alkyl).

In one embodiment, R⁶ is selected from C₁₋₄alkoxyC₁₋₄alkyl, C₁₋₄alkylsulfonyl, COR⁹ (wherein R⁹ is hydrogen or C₁₋₄alkyl), CONRⁱR^{j} (wherein Rⁱ and R^{j} are independently C₁₋₄alkyl) and CH₂NR^{l}R^{m} (wherein R^{l} and R^{m} are independently C₁₋₄alkyl).

In one embodiment, R⁶ is selected from C₁₋₄alkoxyC₁₋₄alkyl, C₁₋₄alkylsulfonyl, C₁₋₄alkylthio and COR⁹.

In one embodiment, R⁶ is selected from C₁₋₂alkoxyC₁₋₂alkyl, C₁₋₂alkylsulfonyl, C₁₋₂alkylthio and COR⁹. In a further embodiment, R⁶ is selected from —COH, —SO₂CH₃, —SCH₃, —C(CH₃)OCH₃, —CH₂CH₂OCH₃ and —CH₂OCH₃.

In one embodiment, R⁶ is selected from —COH, —SO₂CH₃, —SCH₃, —C(CH₃)OCH₃, —CH₂OCH₃, CH₂N(CH₃)₂ and CON(CH₃)₂.

In one embodiment, R¹⁵ is hydrogen.

In one embodiment, R⁷ is selected from hydrogen, C₁₋₂alkyl, C₁₋₂alkoxy, haloC₁₋₂alkyl, haloC₁₋₂alkoxy, halo, cyano, C₁₋₂alkoxyC₁₋₂alkoxy and C₁₋₂alkoxyC₁₋₂alkyl. In a further embodiment, R⁷ is hydrogen.

In one embodiment, R⁸ is hydrogen.

In one embodiment, m is 1. In one embodiment, m is 0.

In one embodiment, there is provided a compound of formula (Ia) or a salt or solvate thereof:

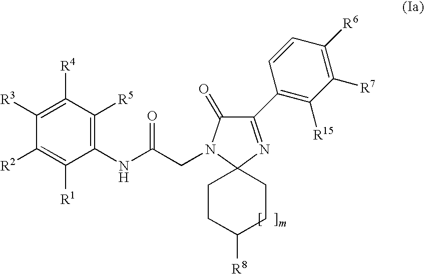

wherein:
R¹ is selected from H, C₁₋₄alkyl, C₁₋₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{a}R^{b} (wherein R^{a} and R^{b} are independently selected from H and C₁-C₄alkyl, or R^{a} and R^{b}, together with the nitrogen atom to which they are attached, form a 4- to 7-membered ring) and cyano;
R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{c}R^{d} (wherein R^{c} and R^{d} are independently selected from H and C₁-C₄alkyl, or R^{c} and R^{d}, together with the nitrogen atom to which they are attached, form a 4- to 7-membered ring) and cyano;
R³ is selected from H, methyl C1-4alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{e}R^{f} (wherein R^{e} and R^{f} are independently selected from H and C₁-C₄alkyl, or R^{e} and R^{f}, together with the nitrogen atom to which they are attached, form a 4- to 7-membered ring) and cyano;
or R² and R³ together form a group selected from —O—CH₂—O— and —O—CH₂—CH₂—O—;
R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{g}R^{h} (wherein R^{g} and R^{h} are independently selected from H and C₁-C₄alkyl, or R^{g} and R^{h}, together with the nitrogen atom to which they are attached, form a 4- to 7-membered ring) and cyano;
R⁵ is selected from hydrogen, chloro, fluoro, C₁-C₄alkyl and CF₃;
R⁶ is selected from C₁₋₄alkoxyC₁₋₄alkyl, C₁₋₄alkylsulfonyl, COR⁹ wherein R⁹ is hydrogen or C₁₋₄alkyl, CONRⁱR^{j} wherein Rⁱ and R^{j} are independently selected from hydrogen, C₁₋₄alkyl or, together with the nitrogen atom to which they are attached, form a 4, 5 or 6-membered ring, or CHR^{k}NR^{l}R^{m} wherein R^{k} is hydrogen or C₁₋₄alkyl and R' and R^{m} are independently selected from hydrogen and C₁₋₄alkyl or R^{l} and R^{m}, together with the nitrogen atom to which they are attached, form a 4, 5 or 6-membered ring;

### R¹⁵ is H or F;

R⁷ is selected from H, C₁-C₄alkyl, C₁-C₄alkoxy, haloC₁-C₄alkyl, haloC₁-C₄alkoxy, halo, cyano, C₁-C₄alkoxyC₁-C₄alkoxy and C₁₋₄alkoxyC₁₋₄alkyl;
R⁸ is selected from hydrogen and methyl; and
m is selected from 0, 1 and 2.

In one embodiment, there is provided a compound of formula (Ib) or a salt thereof:

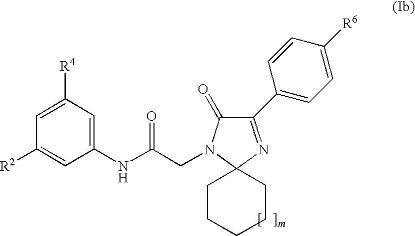

wherein:
R² is selected from hydrogen, C₁₋₄alkyl, C₁₋₄alkoxy, halo, haloC₁₋₄alkyl, haloC₁₋₄alkoxy, C₃₋₆cycloalkyl, C₁₋₄alkylsulfonyl, C₁₋₄alkoxyC₁₋₄alkyl, cyano and CONR^{c}R^{d} (wherein R^{c} and R^{d} are independently selected from H and C₁₋₄alkyl, or R^{c} and R^{d}, together with the nitrogen atom to which they are attached, form a 4- to 7-membered ring);
R⁴ is selected from hydrogen, C₁₋₄alkyl, C₁₋₄alkoxy, halo, haloC₁₋₄alkyl, haloC₁₋₄alkoxy, C₃₋₆cycloalkyl, C₁₋₄alkylsulfonyl, C₁₋₄alkoxyC₁₋₄alkyl, cyano and CONR^{g}R^{h} (wherein R^{g} and R^{h} are independently selected from H and C₁₋₄alkyl, or R^{g} and R^{h}, together with the nitrogen atom to which they are attached, form a 4- to 7-membered ring);
R⁶ is selected from C₁₋₄alkoxyC₁₋₄alkyl, C₁₋₄alkylsulfonyl, COR⁹ (wherein R⁹ is hydrogen or C₁₋₄alkyl), CONRⁱR^{j} (wherein Rⁱ and R^{j} are independently selected from hydrogen and C₁₋₄alkyl or, together with the nitrogen atom to which they are attached, form a 4, 5 or 6-membered ring) and CHR^{k}NR^{l}R^{m} (wherein R^{k} is hydrogen or C₁₋₄alkyl and R^{l} and R^{m} are independently selected from hydrogen and C₁₋₄alkyl or R^{l} and R^{m}, together with the nitrogen atom to which they are attached, form a 4, 5 or 6-membered ring); and
m is selected from 0, 1 and 2.

In one embodiment, there is provided a compound of formula (Ic) or a salt thereof:

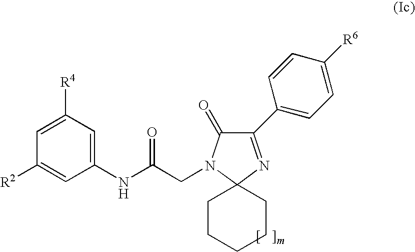

wherein:
R² is selected from halo and haloC₁₋₄alkyl;
R⁴ is selected from hydrogen and halo;
R⁶ is selected from C₁₋₄alkoxyC₁₋₄alkyl, C₁₋₄alkylsulfonyl, COR⁹ (wherein R⁹ is hydrogen or C₁₋₄alkyl), CONRⁱR^{j} (wherein Rⁱ and R^{j} are independently selected from hydrogen and C₁₋₄alkyl or, together with the nitrogen atom to which they are attached, form a 4, 5 or 6-membered ring) and CHR^{k}NR^{l}R^{m} (wherein R^{k} is hydrogen or C₁₋₄alkyl and R^{l} and R^{m} are independently selected from hydrogen and C₁₋₄alkyl or R^{l} and R^{m}, together with the nitrogen atom to which they are attached, form a 4, 5 or 6-membered ring); and
m is selected from 0 and 1.

For the avoidance of doubt, the embodiments of any one feature of the compounds of the invention may be combined with any embodiment of another feature of compounds of the invention to create a further embodiment.

Examples of compounds of the invention include:
1. N-(3,5-difluorophenyl)-2-{3-[4-(methylsulfonyl)phenyl]-2-oxo-1,4-diazaspiro[4.5]dec-3-en-1-yl}acetamide
2. N-(3,5-difluorophenyl)-2-{3-[4-(methylthio)phenyl]-2-oxo-1,4-diazaspiro[4.5]dec-3-en-1-yl}acetamide
3. N-(3,5-difluorophenyl)-2-[3-(4-formylphenyl)-2-oxo-1,4-diazaspiro[4.5]dec-3-en-1-yl]acetamide
4. N-(3,5-difluorophenyl)-2-(3-{4-[(methyloxy)methyl]phenyl}-2-oxo-1,4-diazaspiro[4.5]dec-3-en-1-yl)acetamide
5. N-(3,5-difluorophenyl)-2-(3-{-4-[1-(methyloxy)ethyl]phenyl}-2-oxo-1,4-diazaspiro[4.5]dec-3-en-1-yl)acetamide
   and salts and solvates thereof.
   Further examples include
1. 3-{4-[(Methyloxy)methyl]phenyl}-1-{N-[3-(trifluoromethyl)phenyl]glycyl}-1,4-diazaspiro[4.5]dec-3-en-2-one
2. 3-{4-[(Methyloxy)methyl]phenyl}-1-{N-[3-(trifluoromethyl)phenyl]glycyl}-1,4-diazaspiro[4.4]non-3-en-2-one
3. 1[N-(3,5-Difluorophenyl)glycyl]-3-{4-[(methyloxy)methyl]phenyl}-1,4-diazaspiro[4.4]non-3-en-2-one
   and salts thereof.
   Further examples include
1. N-(3,5-Difluorophenyl)-2-(3-{-4-[1-(methyloxy)ethyl]phenyl}-2-oxo-1,4-diazaspiro[4.4]non-3-en-1-yl)acetamide
2. N-(3,5-Difluorophenyl)-2-(3-{4-[(dimethylamino)methyl]phenyl}-2-oxo-1,4-diazaspiro[4.5]dec-3-en-1-yl)acetamide
3. 4-(4-{2-[(3,5-Difluorophenyl)amino]-2-oxoethyl}-3-oxo-1,4-diazaspiro[4.5]dec-1-en-2-yl)-N,N-dimethylbenzamide
   and salts thereof.

In an embodiment there is provided a compound of formula (I) as defined above or a pharmaceutically acceptable salt thereof.

Salts of compounds of formula (I) which are suitable for use in medicine are those wherein the counterion is pharmaceutically acceptable. However, salts having non-pharmaceutically acceptable counterions are within the scope of the present invention, for example, for use as intermediates in the preparation of other compounds of formula (I) and their pharmaceutically acceptable salts.

As used herein, the term “salt” refers to any salt of a compound according to the present invention prepared from an inorganic or organic acid or base, quaternary ammonium salts and internally formed salts. Pharmaceutically acceptable salts are particularly suitable for medical applications because of their greater aqueous solubility relative to the parent compounds. Such salts must clearly have a pharmaceutically acceptable anion or cation. Suitably pharmaceutically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, (1R)-(−)-10-camphorsulphonic, (1S)-(+)-10-camphorsulphonic, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example naphthalene-1,5-disulphonic, naphthalene-1,3-disulphonic, benzenesulfonic, and p-toluenesulfonic, acids. Salts having a non-pharmaceutically acceptable anion or cation are within the scope of the invention as useful intermediates for the preparation of pharmaceutically acceptable salts and/or for use in non-therapeutic, for example, in vitro, situations. The salts may have any suitable stoichiometry. For example, a salt may have 1:1 or 2:1 stoichiometry. Non-integral stoichiometry ratios are also possible.

Solvates of the compounds of formula (I) and solvates of the salts of the compounds of formula (I) are included within the scope of the present invention. As used herein, the term “solvate” refers to a complex of variable stoichiometry formed by a solute (in this invention, a compound of formula (I) or a salt thereof) and a solvent. Those skilled in the art of organic chemistry will appreciate that many organic compounds can form such complexes with solvents in which they are reacted or from which they are precipitated or crystallized. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water. Where the solvent used is water such a solvate may then also be referred to as a hydrate.

Hereinafter, compounds of formula (I) (whether in solvated or unsolvated form) or their pharmaceutically acceptable salts (whether in solvated or unsolvated form) or prodrugs thereof defined in any aspect of the invention (except intermediate compounds in chemical processes) are referred to as “compounds of the invention”.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms), for example when R⁸ in formula (I) above is methyl and n is 0; or if R⁶ contains an asymmetric carbon. The individual stereoisomers (enantiomers and diastereoisomers) and mixtures of these are included within the scope of the present invention. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994). Likewise, it is understood that compounds of formula (I) may exist in tautomeric forms other than that shown in the formula and these are also included within the scope of the present invention.

In one embodiment, an optically pure enantiomer of a compound of the present invention is provided. The term “optically pure enantiomer” means that the compound contains greater than about 90% of the desired isomer by weight, such as greater than about 95% of the desired isomer by weight, or greater than about 99% of the desired isomer by weight, said weight percent based upon the total weight of the isomer(s) of the compound.

Compounds of general formula (I) may be prepared by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I).

Typical reaction routes for the preparation of a compound of formula (I) as hereinbefore defined, are shown below.

Compounds of formula (I) can be prepared by reacting a compound of formula (II) with a base, for example sodium hydride, in a suitable inert solvent, for example dimethylformamide, followed by treatment with a compound of formula (III) where X is halogen as shown in Scheme 1.

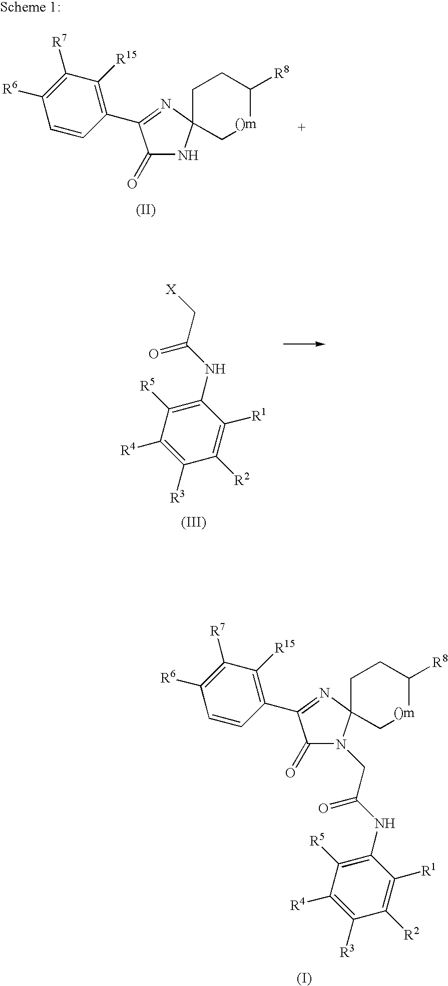

Compounds of formula (III) can be prepared by standard methods, for example as shown in Scheme 2. For example, an aniline of formula (IV) may be combined with an haloacetyl halide of formula (XII) where X and X′ are halogen, for example chloroacetyl chloride or bromoacetyl bromide in an inert solvent, for example, dioxan and heated to give a compound of formula (III).

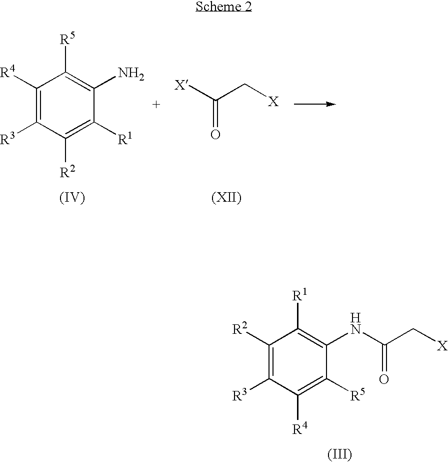

Compounds of formula (II) may be prepared by desulphurisation of compounds of formula (V) using an oxidising agent, for example hydrogen peroxide as shown for example in Scheme 3.

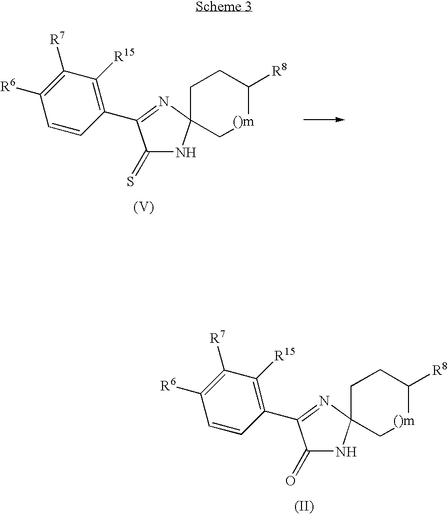

Compounds of formula (V) can be prepared by treating a ketothioamide of formula (VI) with the appropriate ketone of formula (VII) in the presence of a source of ammonia, for example ammonium acetate as shown in Scheme 4. In one embodiment, this reaction is performed in a solvent, for example isopropanol, at room or elevated temperature, preferably elevated temperature, for example at reflux.

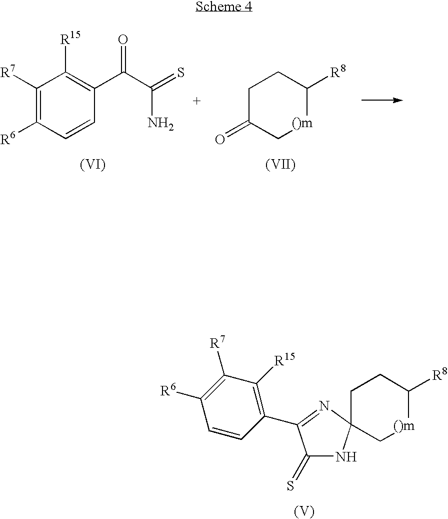

Thioamides of formula (VI) can be prepared from acylnitriles of formula (VIII) by treating with, for example hydrogen sulphide in the presence of an organic base, for example triethylamine in an inert solvent, for example diethyl ether at room temperature. Acylnitriles of formula (VIII) can be prepared from the appropriate acid chloride (IX) and a source of cyanide, conveniently copper (I) cyanide, at elevated temperatures, for example greater than 150° C. for example in the absence of solvent.

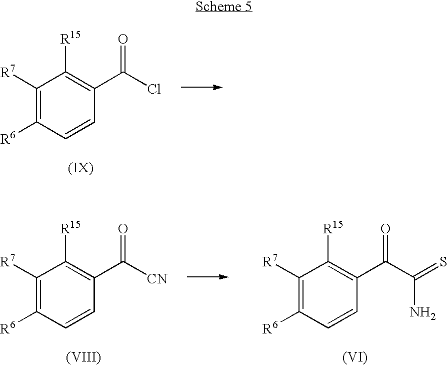

Alternatively, compounds of formula (II) can be synthesised as shown in Scheme 6.

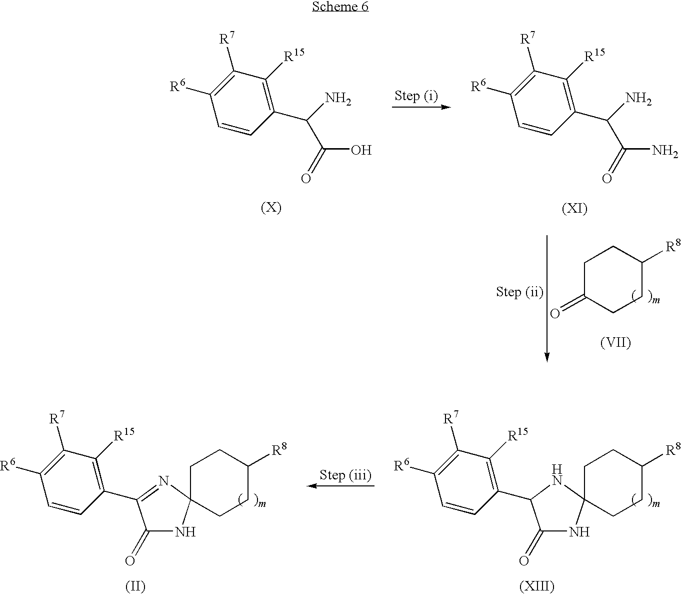

wherein R⁶, R⁷, R⁸ and R¹⁵ are as defined for formula (I).

The arylglycine of formula (X) can be converted, step (i), to the corresponding arylglycinamide of formula (XI) by standard methods, for example, by reaction of compounds of formula (X) with thionyl chloride or acetyl chloride in methanol, followed by subsequent reaction of the intermediate methyl ester hydrochloride with aqueous ammonia.

Arylglycinamides of formula (XI) can be converted to compounds of formula (XIII), step (ii), by condensation with ketones of formula (VII), for example, by heating in an inert solvent such as methanol, in the presence or absence of a catalyst such as H—Y zeolites.

Oxidation of compounds of formula (XIII), step (iii), to afford compounds of formula (II) can be achieved by methods known in the art, for example, by reaction with N-bromosuccinimide in an inert solvent, such as dichloromethane.

Compounds of formula (X) are known in the literature or can be prepared as shown in Scheme 7 wherein R⁶, R⁷, R¹⁵ are as defined in formula (I), except when R⁷ is halo.

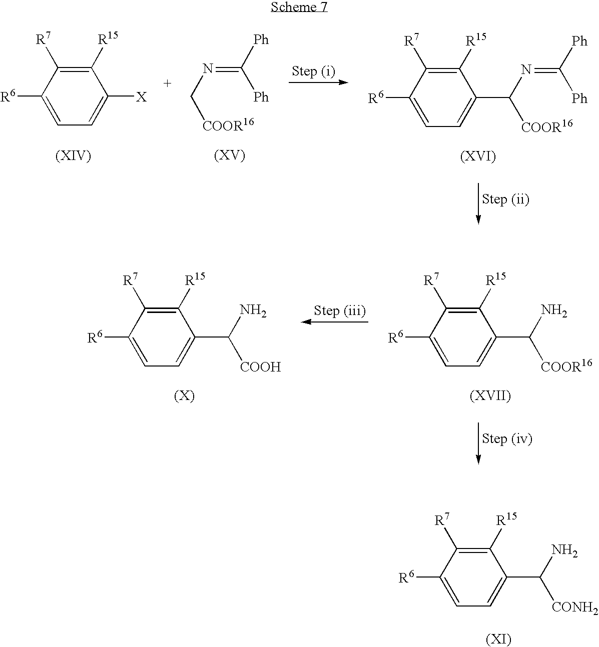

For example a compound of formula (XIV), where X is a halogen, can be treated with a N-(diphenylmethylidene)glycinate ester (XV), where R¹⁶ is lower alkyl such as methyl or ethyl, in the presence of a palladium catalyst such as bis(tri-t-butylphosphine)palladium (0) and a base such as potassium phosphate in a solvent such as toluene at elevated temperature, step (i), to give a compound of formula (XVI). Mild acidic hydrolysis of the imine, step (ii), for example using dilute HCl acid at room temperature can afford the glycine ester (XVII), whereas the glycine (X) can be prepared by more extensive hydrolysis, step (iii). Treatment of ester (XVII) with aqueous ammonia can give the glycinamide (XI), step (iv).

Alternatively compounds of formula (II) can be synthesised as shown in scheme 8 wherein R⁶, R⁷, R⁸, R¹⁵ and m are as defined in formula (I).

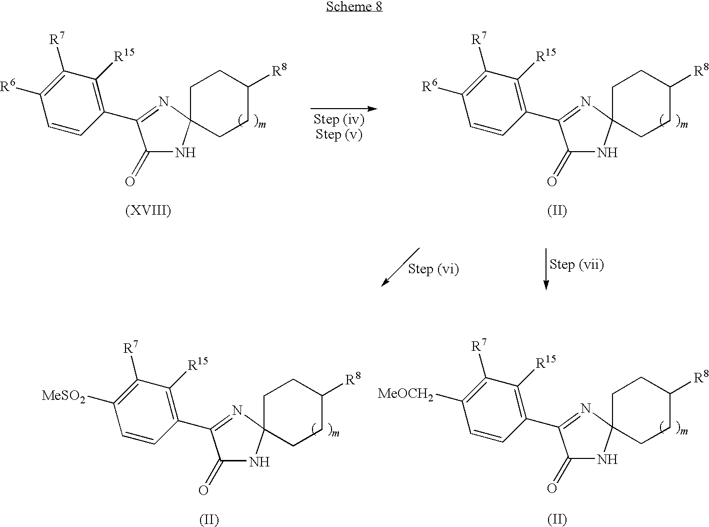

Metal halogen exchange step (iv) of a compound of formula (XVIII) by treatment with an alkyl lithium, for example, n-butyl lithium, sec-butyl lithium or tert-butyl lithium in a solvent such as tetrahydrofuran or diethyl ether at or preferably a temperature lower than room temperature. Subsequently, in situ treatment of the generated intermediate with an appropriate electrophilic reagent step (v) such as dimethyl disulphide, dimethylformamide, formaldehyde, acetaldehyde or chloromethyl methyl ether will provide, either directly, desired compounds of formula (II) or compounds of formula (II) that may be converted to other compounds of formula (II) by standard techniques. For example, step (vi), a compound of formula (II) R⁶ is SMe can be oxidised to a compound of formula (II) where R⁶ is SO₂Me by treatment with meta-chloroperbenzoic acid in an inert solvent such as dichloromethane or with sodium periodate in methanol/water. Or, for example step (vii), a compound of formula (II) where R⁶ is CH₂OH, can be converted, by treatment with an activating agent such as methanesulphonyl chloride, in the presence of a base such as triethylamine in an inert solvent such as dichloromethane at ambient or reduced temperature and subsequent treatment with an alcohol such as methanol to another compound of formula (II)

Alternatively compounds of formula (II) can be prepared as shown in scheme 9 wherein R⁶, R⁷, R⁸ and R¹⁵ are as defined in formula (I) by application of palladium mediated chemistry.

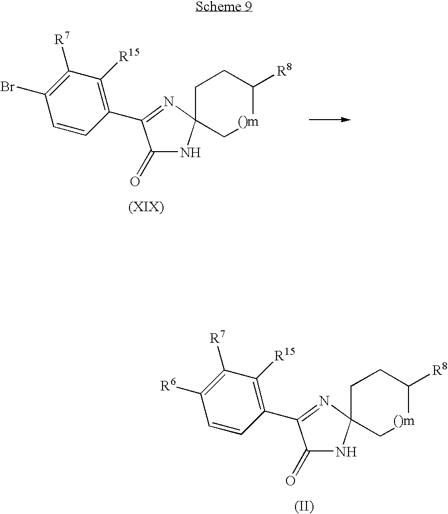

For example, treatment of a compound of structure (XIX) with an appropriate palladium catalyst such as tetrakis(triphenylphosphine)palladium[0] or palladium acetate in conjunction with a phosphine ligand such as 1,3-(bis)triphenylphosphino)propane, a base such as sodium carbonate, triethylamine or diisopropylamine and a reagent that may undergo palladium mediated insertion to an aromatic ring such as carbon monoxide in the presence of a secondary amine such as N,N-dimethylamine to give a compound of formula (II) where R⁶ is CONMe₂ or an olefin such as butylvinyl ether to give, after appropriate work-up, a compound where R⁶ is COMe. These reactions may be performed in a range of solvents including tetrahydrofuran, dimethylformamide, dioxan or dimethylsulphoxide or combinations of solvents, optionally in the presence of an ionic liquid such as 1-butyl-3-imidazolium tetrafluoroborate either at ambient or preferably elevated temperatures. These compounds may be further transformed to generate additional compounds of formula (II) by application of standard chemical transformations.

Compounds of formula (II) can also be converted to compounds of formula (I) as shown in Scheme 10.

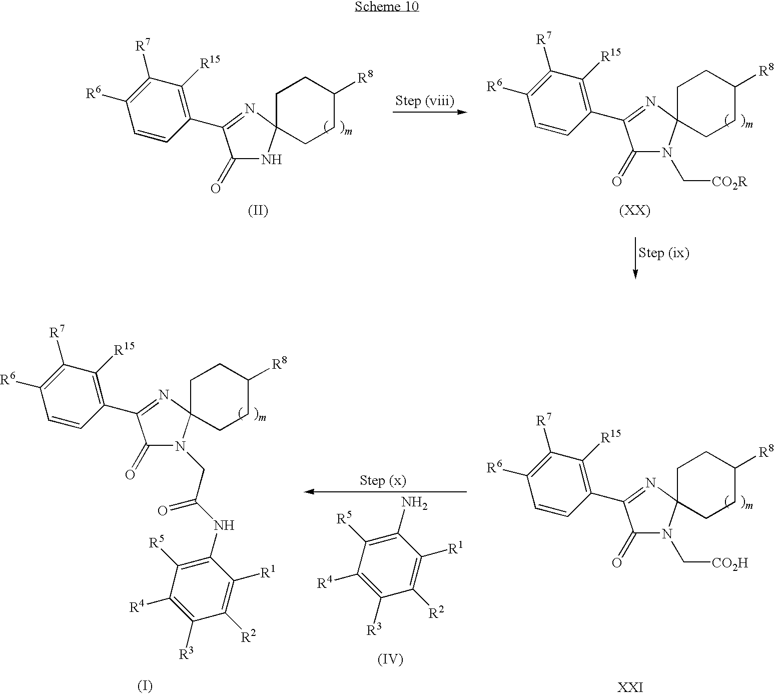

wherein R¹, R², R³, R⁴, R⁵, R⁶, R⁷, R⁸ and R¹⁵ are as defined for compounds of formula (I).

Compounds of formula (XX) can be prepared using standard methods from compounds of formula (II), step (viii), for example, by reaction with an appropriate haloester in the presence of a base, such as sodium hydride or potassium carbonate, in a suitable inert solvent, such as dimethylformamide, at room temperature or elevated temperature as appropriate.

Removal of the ester group R from compounds of formula (XX) to afford the acids of formula (XXI), step (ix), can be achieved by known methods, for example by use of a base, such as sodium hydroxide, in an inert solvent, such as aqueous methanol or aqueous ethanol, with or without heating as appropriate.

Compounds of formula (XXI) can be converted to compounds of formula (I), step (x), by reaction with an aniline of formula (IV) using a variety of methods known in the art. For example, the acylation step (x) can be achieved by reaction of the acid (XXI) with an aniline of formula (IV), in an inert solvent, such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), or O-(7-azabenzotriazol-1-yl)-1,1,3,3-tetramethyluronium hexafluoro phosphate (HATU). Alternatively, compounds of formula (XXI) are converted to compounds of formula (XXII):

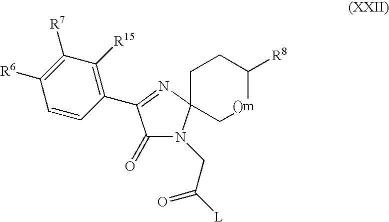

wherein R⁶, R⁷, R⁸ and R¹⁵ are as defined in formula (I) and L represents a suitable leaving group. Examples of leaving groups include halogen, OC(═O)alkyl, OC(═O)O-alkyl and OSO₂Me. L may be halogen and acylation in step (x) may be carried out in an inert solvent such as dichloromethane, in the presence of a base, such as triethylamine.

Within these schemes there is scope to convert a group R¹ into another group R¹ and similarly for groups R², R³, R⁴, R⁵, R⁶, R⁷ and R¹⁵. For example, scheme 11(a) shows a compound of formula (I) where R⁶ is COR⁹ wherein R⁹ is hydrogen or C₁₋₄alkyl may be converted to compounds of formula (I) wherein R⁶ is C₁₋₄alkoxyC₁₋₄alkyl, by treatment with a reducing agent for example sodium borohydride in an appropriate solvent such as methanol, or by treatment with an alkylmagnesium halide in suitable solvent such as tetrahydrofuran, to form the alcohol; followed by treatment with an activating agent such as methanesulphonyl chloride, in the presence of a base such as triethylamine in an inert solvent such as dichloromethane at ambient or reduced temperature and subsequent treatment with a C₁₋₄alkoxide in the corresponding alcohol solution.

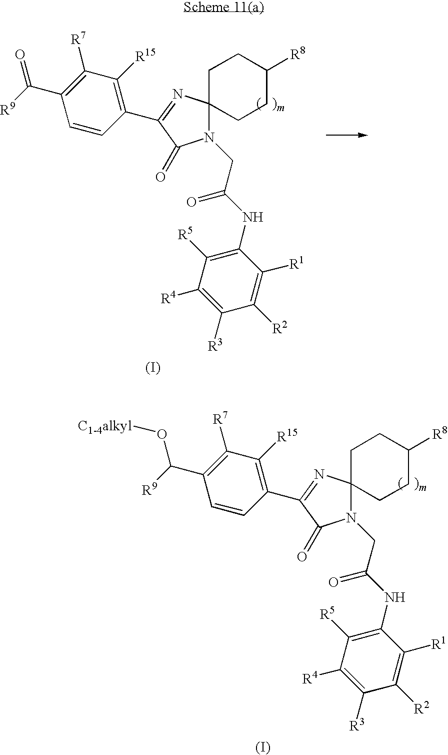

Scheme 11(b) shows that a compound of formula (I) where R⁶ is SMe can be oxidised to a compound of formula (I) where R⁶ is SO₂Me by treatment with meta-chloroperbenzoic acid in an inert solvent such as dichloromethane or with sodium periodate in methanol/water.

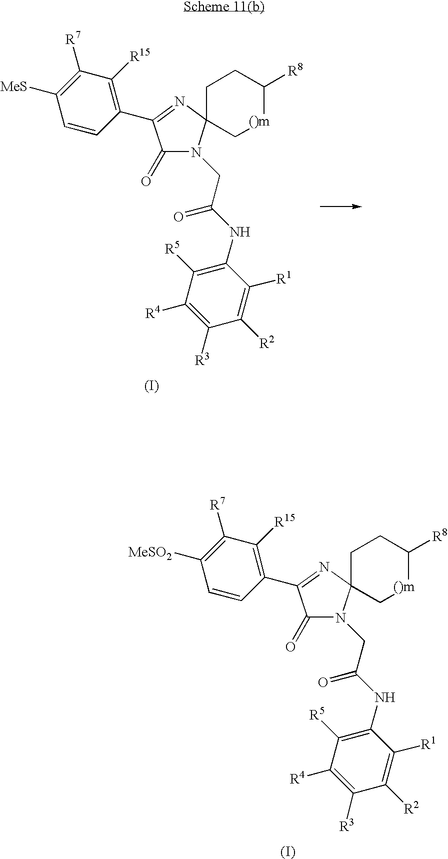

Salts may be prepared conventionally by reaction with the appropriate acid or acid derivative.

In one aspect, the present invention provides a process for the preparation of a compound of the invention, the process comprising

(a) reacting a compound of formula (II):

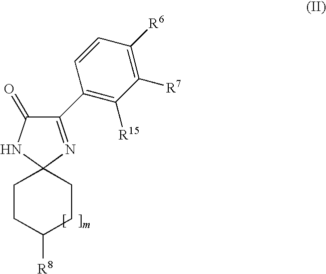

wherein m, R⁶, R⁷, R⁸ and R¹⁵ are as defined for formula (I), with a base in a suitable inert solvent, followed by treatment with a compound of formula (III):

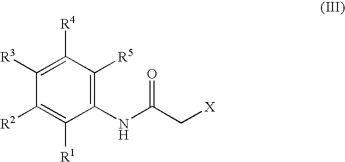

wherein R¹, R², R³, R⁴ and R⁵ are as defined for formula (I), and X is a leaving group; or
(b) reacting a compound of formula (XXI):

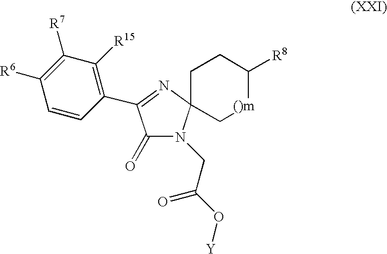

wherein m, R⁶, R⁷, R⁸ and R¹⁵ are as defined for formula (I) and Y is hydrogen or a leaving group; with a compound of formula (IV):

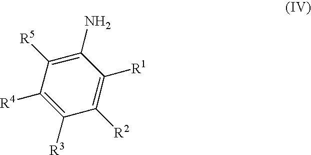

wherein R¹, R², R³, R⁴ and R⁵ are as defined for formula (I);
(c) for compounds of formula (I) wherein R6=alkoxyalkyl and salts thereof, treating a compound

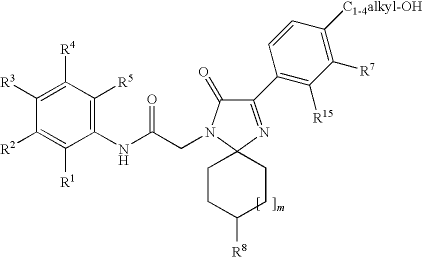

with an activating agent such as methanesulphonyl chloride in presence of suitable base and solvent, followed by treatment with a C₁₋₄alkoxide in a suitable solvent, such as the corresponding C₁₋₄ alcohol;
and thereafter optionally:
(i) forming a salt or solvate; and/or
(ii) converting a compound of formula (I) into another compound of formula (I).

In process (c), a suitable activating is methanesulphonyl chloride. A suitable solvent is the corresponding C₁₋₄alcohol.

In one aspect, there is provided a compound of formula (II) or a salt thereof:

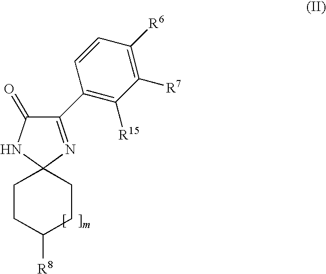

wherein:
R⁶ is selected from C₁₋₄alkoxyC₁₋₄alkyl, C₁₋₄alkylsulfonyl, COR⁹ (wherein R⁹ is hydrogen or C₁₋₄alkyl), CONRⁱR^{j} (wherein Rⁱ and R^{j} are independently selected from hydrogen and C₁₋₄alkyl or, together with the nitrogen atom to which they are attached, form a 4, 5 or 6-membered ring) and CHR^{k}NR^{l}R^{m} (wherein R^{k} is hydrogen or C₁₋₄alkyl and R^{l} and R^{m} are independently selected from hydrogen and C₁₋₄alkyl or R^{l} and R^{m}, together with the nitrogen atom to which they are attached, form a 4, 5 or 6-membered ring);
R¹⁵ is hydrogen or fluoro;
R⁷ is selected from hydrogen, C₁₋₄alkyl, C₁₋₄alkoxy, haloC₁₋₄alkoxy, halo, cyano, C₁₋₄alkoxyC₁₋₄alkoxy and C₁₋₄alkoxyC₁₋₄alkyl;
R⁸ is selected from hydrogen and methyl; and
m is selected from 0, 1 and 2.

In one aspect, there is provided 3-{4-[(Methyloxy)methyl]phenyl}-1,4-diazaspiro[4.5]dec-3-en-2-one and salts thereof.

The compounds of the present invention inhibit the GlyT1 transporter, as measured by the assay below. Such compounds are therefore of potential utility for the treatment of certain neurological and neuropsychiatric disorders. The compounds may selectively inhibit the GlyT1 transporter over the GlyT2 transporter. Some compounds of the invention may have mixed GlyT1/GlyT2 activity.

The affinities of the compounds of this invention for the GlyT1 transporter can be determined by the following assay. In the assays used herein, the compounds of the present invention were not necessarily from the same batch described above. The test compound made in one batch may have been combined with other batch(es) for the assay(s).

HEK293 cells expressing the Glycine (Type 1) transporter are grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks are harvested and resuspended at 4×10⁵ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds are serially diluted 2.5-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) is added to the cell suspension and 5 μL of the cell/bead suspension transferred to each well of a 384-well white solid bottom plate (1,000 cells/well) containing 100 nL of test compounds. Substrate (5 μL) is added to each well [1:100 dilution of [³H]-glycine stock in assay buffer containing 2.5 μM glycine). Final DMSO concentration was 1% v/v. Data was collected using a Perkin Elmer Viewlux. pIC₅₀ values were determined using ActivityBase.

Compounds are considered to have activity at the GlyT1 transporter if they have a pIC₅₀ of 5.0 or above. The example compounds below were found to have an average pIC₅₀ at the GlyT1 transporter of equal to or greater than 5.8.

As used herein, the term “disorders mediated by GlyT1” refers to disorders that may be treated by the administration of a medicament that alters the activity of the GlyT1 transporter. These include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury. Other disorders include Parkinson's disease, dyskinetic disorders, cognitive impairment, emesis, movement disorders, amnesia, circadian rhythm disorders, aggression and vertigo.

In one embodiment, the disorder mediated by GlyT1 to be treated by the use or method as hereinbefore described is a psychosis, including schizophrenia, dementia and attention deficit disorders. In one embodiment, the disorder is schizophrenia.

As used herein, the term “effective amount” means that amount of a drug or pharmaceutical agent that will elicit the biological or medical response of a tissue, system, animal or human that is being sought, for instance, by a researcher or clinician.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). Treatment of the various subtypes of the disorders mentioned herein using the compounds of the invention are contemplated as part of the present invention. Numbers in brackets after the listed diseases below refer to the classification code in DSM-IV.

In particular, the compounds of the invention be of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of the invention may be also of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of the invention may also be of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of the invention may also be of use in the treatment of substance-related disorders including Substance Use Disorders such as Substance Dependence and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of the invention may also be of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of the invention may also be of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of the invention may also be of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of the invention may also be of use in the treatment of Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).

The compounds of the invention may also be of use in the treatment of cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of the present invention may also be of use for the treatment of cognition impairment which arises in association or as a result of other diseases such as schizophrenia, bipolar disorder, depression, other psychiatric disorders and psychotic conditions associated with cognitive impairment.

The compounds of the invention may also be of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The compounds of the invention may also be of use as anticonvulsants. The compounds of the invention are thus useful in the treatment of convulsions in mammals, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures. The invention also provides a method of treating convulsions, which comprises administering to a mammal in need thereof an effective amount of a compound of the invention as hereinbefore described or a salt thereof. Treatment of epilepsy may be carried out by the administration of a non-toxic anticonvulsant effective amount of a compound of the formula (I) or a salt thereof.

The compounds of the invention may also be of use in the treatment of neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

As used herein, the terms “treatment” and “treating” refer to the alleviation and/or cure of established symptoms as well as prophylaxis.

The invention thus provides compounds of formula (I) and salts thereof for use in therapy.

The invention also provides compounds of formula (I) and salts thereof for use in the treatment of a disorder mediated by GlyT1.

In a further aspect of the present invention, there is provided a method of treating a disorder mediated by GlyT1 comprising administering a compound of formula (I) or a salt thereof.

In a further aspect of the present invention there is provided the use of a compound of formula (I) or a salt thereof in the manufacture of a medicament for use in the treatment of a disorder mediated by GlyT1.

In order to use a compound of the present invention in therapy, it will normally be formulated into a pharmaceutical composition in accordance with standard pharmaceutical practice. The present invention also provides a pharmaceutical composition, which comprises a compound of formula (I) or a salt thereof and at least one pharmaceutically acceptable excipient.

In a further aspect, the present invention provides a process for preparing a pharmaceutical composition, the process comprising mixing a compound of formula (I) or a salt thereof and at least one pharmaceutically acceptable excipient.

A pharmaceutical composition of the invention is usually adapted for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound. In one embodiment, oral administration is provided.

Compositions suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Compositions suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Compositions suitable for parenteral administration typically comprise sterile aqueous solutions containing a predetermined concentration of the active compound; the solution may be isotonic with the blood of the intended recipient. Such solutions may be administered intravenously or by subcutaneous or intramuscular injection.

Compositions suitable for rectal administration may be provided as unit-dose suppositories comprising the active ingredient and one or more solid carriers forming the suppository base, for example, cocoa butter.

Compositions suitable for topical or intranasal application include ointments, creams, lotions, pastes, gels, sprays, aerosols and oils. Suitable carriers for such compositions include petroleum jelly, lanolin, polyethylene glycols, alcohols, and combinations thereof.

The compositions of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

A proposed dose of the active ingredient for use according to the invention for oral, sub-lingual, parenteral, buccal, rectal, intranasal or topical administration to a human (of approximately 70 kg bodyweight) for the treatment of neurological and neuropsychiatric disorders mediated by a GlyT1 inhibitor, including schizophrenia, may be about 0.1 to about 1000 mg, for example about 0.5 mg to about 1000 mg, or about 1 mg to about 1000 mg, or about 5 mg to about 500 mg, or about 10 mg to about 100 mg of the active ingredient per unit dose, which could be administered, for example, 1 to 4 times per day.

The compounds of formula (I) and their salts thereof may also be suitable for combination with other therapeutic agents, such as typical and atypical antipsychotics. Thus, the present invention also provides:
i) a combination comprising a compound of formula (I) with one or more further therapeutic agents such an one or more antipsychotic;
ii) a pharmaceutical composition comprising a combination product as defined in i) above and at least one carrier, diluent or excipient;
iii) the use of a combination as defined in i) above in the manufacture of a medicament for treating or preventing a disease or condition caused by a reduction or imbalance in glutamate receptor function in a mammal;
iv) a combination as defined in i) above for use in treating or preventing a disease or condition caused by a reduction or imbalance in glutamate receptor function in a mammal;
v) a kit-of-parts for use in the treatment of a psychotic disorder comprising a first dosage form comprising a compound of the invention and one or more further dosage forms each comprising a antipsychotic agent for simultaneous therapeutic administration.
vi) a combination as defined in i) above for use in therapy;
vii) a method of treatment or prevention of a disease or condition caused by a reduction or imbalance in glutamate receptor function in a mammal comprising administering an effective amount of a combination as defined in i) above.

The combination therapies of the invention may be administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a salt thereof and at least one antipsychotic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, the compounds of formula (I) or a salt thereof may be administered as adjunctive therapeutic treatment to patients who are receiving administration of at least one antipsychotic agent, but the scope of the invention also includes the adjunctive therapeutic administration of at least one antipsychotic agent to patients who are receiving administration of compounds of formula (I) or a salt thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a salt thereof to a patient receiving therapeutic administration of at least one antipsychotic agent. In a further aspect, the invention provides the use of compounds of formula (I) or a salt thereof in the manufacture of a medicament for adjunctive therapeutic administration for the treatment of a psychotic disorder in a patient receiving therapeutic administration of at least one antipsychotic agent. The invention further provides compounds of formula (I) or a salt thereof for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of compounds of formula (I) or a salt thereof. In a further aspect, the invention provides the use of at least one antipsychotic agent in the manufacture of a medicament for adjunctive therapeutic administration for the treatment of a psychotic disorder in a patient receiving therapeutic administration of compounds of formula (I) or a salt thereof. The invention further provides at least one antipsychotic agent for adjunctive therapeutic administration for the treatment of a psychotic disorder in a patient receiving therapeutic administration of compounds of formula (I) or a salt thereof.

In a further aspect, the invention provides a method of treatment of a psychotic disorder by simultaneous therapeutic administration of compounds of formula (I) or a salt thereof in combination with at least one antipsychotic agent. The invention further provides the use of a combination of compounds of formula (I) or a salt thereof and at least one antipsychotic agent in the manufacture of a medicament for simultaneous therapeutic administration in the treatment of a psychotic disorder. The invention further provides the use of compounds of formula (I) or a salt thereof in the manufacture of a medicament for simultaneous therapeutic administration with at least one antipsychotic agent in the treatment of a psychotic disorder. The invention further provides compounds of formula (I) or a salt thereof for use for simultaneous therapeutic administration with at least one antipsychotic agent in the treatment of a psychotic disorder. The invention further provides the use of at least one antipsychotic agent in the manufacture of a medicament for simultaneous therapeutic administration with compounds of formula (I) or a salt thereof in the treatment of a psychotic disorder.

In further aspects, the invention provides a method of treatment of a psychotic disorder by simultaneous therapeutic administration of a pharmaceutical composition comprising compounds of formula (I) or a salt thereof and at least one mood stabilising or antimanic agent, a pharmaceutical composition comprising compounds of formula (I) or a salt thereof and at least one mood stabilising or antimanic agent, the use of a pharmaceutical composition comprising compounds of formula (I) or a salt thereof and at least one mood stabilising or antimanic agent in the manufacture of a medicament for the treatment of a psychotic disorder, and a pharmaceutical composition comprising compounds of formula (I) or a salt thereof and at least one mood stabilising or antimanic agent for use in the treatment of a psychotic disorder.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs are as follows: clozapine (available under the tradename CLOZARIL®, from Mylan, Zenith Goldline, UDL, Novartis); olanzapine (available under the tradename ZYPREX®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); thiothixene (available under the tradename NAVANE®;, from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman; perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used. Other antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), ziprasidone, and hoperidone.

It will be appreciated by those skilled in the art that the compounds according to the invention may advantageously be used in conjunction with one or more other therapeutic agents, for instance, antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

The invention is further illustrated by the following non-limiting examples.

The starting material may not necessarily have been prepared from the batch detailed in the relevant Description. All quoted retention times are as measured using LC/MS (Liquid Chromatography/Mass Spectrometry). Where appropriate, these retention times were used as a guide for purification using mass-directed auto-preparation (MDAP), which refers to purification by HPLC, wherein fraction collection is triggered by detection of the programmed mass ion for the compound of interest.

Where reactions are described as having been carried out in a similar manner to earlier, more completely described reactions, the general reaction conditions used were essentially the same. Work up conditions used were of the types standard in the art, but may have been adapted from one reaction to another.

Starting materials were obtained from commercial suppliers and used without further purification unless otherwise stated. Flash chromatography was carried out using pre-packed Isolute Flash™ or Biotage™ silica-gel columns as the stationary phase and analytical grade solvents as the eluent unless otherwise stated.

NMR spectra were obtained at between 294 and 296K at 400 MHz frequency using either a Bruker™ DPX400 or AV400 machine and run as a dilute solution of CDCl₃ unless otherwise stated. All NMR spectra were referenced to tetramethylsilane (TMS δ_{H} 0, δ_{C} 0). All coupling constants are reported in hertz (Hz), and multiplicities are labelled s (singlet), bs (broad singlet), d (doublet), t (triplet), q (quartet), dd (doublet of doublets), dt (doublet of triplets) and m (multiplet).

Total ion current traces were obtained for electrospray positive and negative ionisation (ES+/ES−) and/or atmospheric pressure chemical positive and negative ionisation (AP+/AP−).

Unless otherwise stated, all compounds with chiral centre(s) are racemic.

### Abbreviations:

THF tetrahydrofuran
DCM dichloromethane
DMF dimethylformamide
DMSO dimethyl sulfoxide
g grams
iPrOH isopropyl alcohol
ml millilitres
mmol millimoles
EtOAc ethyl acetate

### Analytical LC/Ms Chromatography Conditions:

| | |
|---|---|
| Column: | Waters Atlantis 50 mm × 4.6 mm, 3 um |
| | particle size |
| Mobile phase: | A: 0.05% Formic acid + Water |
| | B: Acetonitrile + 0.05% Formic acid |
| Gradient: | 5-min runtime: 3% B to 97% B over 4 min |
| Flow rate: | 3 ml/min |
| UV wavelength range: | 220-330 nm |
| Temperature: | 30° C. |

### Mass Directed Auto-Purification System Chromatography Conditions:

| | |
|---|---|
| Column: | Waters Atlantis 19 mm × 100 mm or |
| | 30 mm × 100 mm, 5um |
| | particle size |
| Mobile phase: | A: 0.1% Formic acid + Water |
| | B: Acetonitrile + 0.1% Formic acid |
| Flow rate: | 20 or 40 ml/min |

There are five methods used depending on the analytical retention time of the compound of interest. They have a 13.5-minute runtime, which comprises of a 10-minute gradient followed by a 3.5 minute column flush and re-equilibration step. (i) 1.0-1.5 mins=5-30% B; (ii) 1.5-2.2=15-55% B; (iii) 2.2-2.9=30-85% B; (iv) 2.9-3.6 mins=50-99% B; (v) 3.6-5.0 mins=80-99% B (in 6 minutes followed by 7.5 minutes flush and re-equilibration).

### Description 1: 2-Amino-2-(4-bromophenyl)acetamide

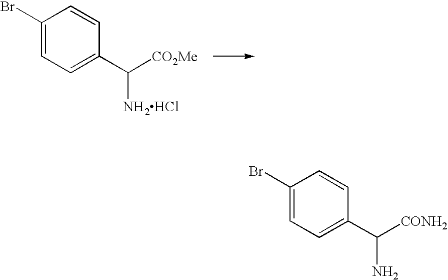

Methyl amino(4-bromophenyl)acetate hydrochloride (commercially available from Bionet Research) (5.0 g; 17.82 mmol) was dissolved in 0.88 ammonia (75 ml; ca. 1.1 mol) and stirred under argon at room temperature for 16 h. The reaction mixture was extracted 4 times with DCM, the organic solution was washed with brine, the extracts dried (MgSO₄) and evaporated. Trituration of the residue with hexane gave the title product as a white solid (2.69 g; 66%). ¹H NMR (d₆-DMSO) δ: 2.19 (2H, br s), 4.27 (1H, s), 7.06 (1H, br s), 7.36 (2H, d), 7.50 (3H, d).

### Description 2: 3-(4-Bromophenyl)-1,4-diazaspiro[4.5]decan-2-one

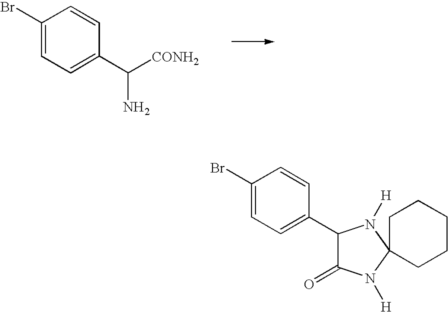

A mixture of 2-amino-2-(4-bromophenyl)acetamide (2.69 g; 11.75 mmol, Description 1), cyclohexanone (1.22 ml; 1 eq; 11.75 mmol) and H—Y zeolites (2.69 g) in methanol (100 ml) was stirred vigorously with heating under argon at 80° C. for 16 h. After cooling, the mixture was filtered through Celite, washing well with methanol. The filtrate was evaporated at reduced pressure to give the title product (2.22 g; 61%). ¹H NMR (d₆-DMSO) δ: 1.30-1.37 (2H, m), 1.50-1.62 (8H, m), 3.50 (1H, d), 4.56 (1H, d), 7.43 (2H, d), 7.51 (2H, d), 8.63 (1H, s). Mass Spectrum (Electrospray LC/MS): Found 309 & 311 (MH⁺). C₁₄H₁₇⁷⁹BrN₂O requires 308 and C₁₄H₁₇⁸¹BrN₂O requires 310. Ret. time 2.21 min.

### Description 3: 3-(4-Bromophenyl)-1,4-diazaspiro[4.5]dec-3-en-2-one

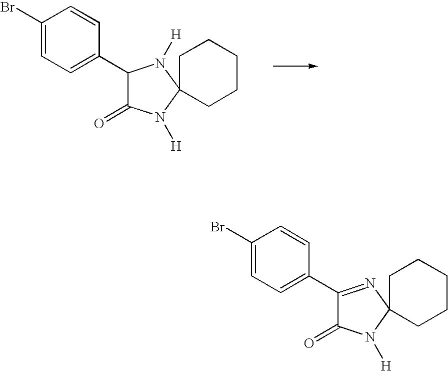

3-(4-bromophenyl)-1,4-diazaspiro[4.5]decan-2-one (2.22 g; 7.19 mmol, Description 2) was dissolved in DCM (50 ml) and stirred at room temperature for 16 hours under an atmosphere of argon with N-bromosuccinimide (1.29 g; 1 eq; 7.19 mmol) A solution of saturated sodium hydrogen carbonate (100 ml) was then added and stirring continued for 1 hour at room temperature. The organic layer was separated, dried (MgSO₄) and evaporated at reduced pressure. The residue was triturated with hexane and filtered to yield the title compound (2.18 g; 99%) as a yellow solid. ¹H NMR (d₆-DMSO) δ: 1.42-1.88 (10H, m), 7.71 (2H, d), 8.28 (2H, d), 10.30 (1H, br s). Mass Spectrum (Electrospray LC/MS): Found 307 & 309 (MH⁺). C₁₄H₁₆⁷⁹BrN₂O requires 306 and C₁₄H₁₆⁸¹BrN₂O requires 308. Ret. time 3.08 min.

### Description 4. 4-(3-Oxo-1,4-diazaspiro[4.5]dec-1-en-2-yl)benzaldehyde

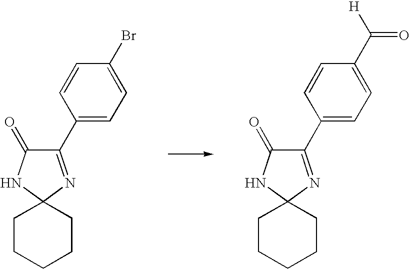

3-(4-Bromophenyl)-1,4-diazaspiro[4.5]dec-3-en-2-one (5.0 g; 16.29 mmol, Description 3) was dissolved in dry tetrahydrofuran (200 ml) and cooled to −78° C., with stirring, under an atmosphere of argon. A solution of n-butyl lithium in hexane (19.5 ml; 3 eq; 2.5M solution; 48.75 mmol) was added dropwise to the stirred solution and stirring was continued at −78° C. for 30 minutes. A solution of N,N-dimethylformamide (3.76 ml; 3 eq; 48.87 mmol) in dry tetrahydrofuran (5 ml) was added and the reaction solution was stirred at −78° C. for 2 hours. The reaction was then quenched by the addition of saturated ammonium chloride solution and allowed to reach room temperature. The reaction solution was then partitioned between ethyl acetate and water. The organic solution was dried (MgSO₄) and evaporated at reduced pressure. The residue was chromatographed over silica gel (50 g). Elution with a gradient of 0 to 50% ethyl acetate in hexane provided the title compound as a white solid (0.762 g). ¹H NMR (CDCl₃) δ: 1.50-2.03 (10H, m), 7.98 (2H, d), 8.12 (1H, broad s), 8.59 (2H, d), 10.10 (1H, s). Mass Spectrum (Electrospray LC/MS): Found 257 (MH⁺). C₁₅H₁₆N₂O₂ requires 256. Ret. time 2.47 min.

### Description 5. N-(3,5-Difluorophenyl)-2-{3-[4-(hydroxymethyl)phenyl]-2-oxo-1,4-diazaspiro[4.5]dec-3-en-1-yl}acetamide

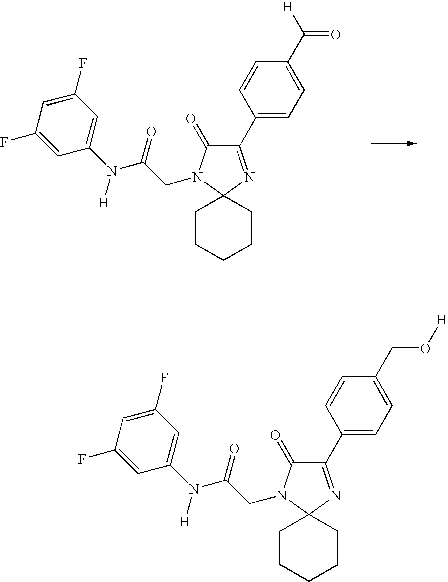

N-(3,5-Difluorophenyl)-2-[3-(4-formylphenyl)-2-oxo-1,4-diazaspiro[4.5]dec-3-en-1-yl]acetamide (271 mg; 0.637 mmol, Example 1) was dissolved in methanol (10 ml) and sodium borohydride (25 mg, 1 eq, 0.637 mmol) was added to the stirred solution. Stirring was continued at room temperature under an atmosphere of argon for 2 h. The reaction solution was then partitioned between ethyl acetate and water. The organic solution was washed with brine, dried (MgSO₄), and evaporated at reduced pressure to yield the title compound as a white solid (200 mg). ¹H NMR (CDCl₃) δ: 1.30-1.48 (3H, broad d), 1.75-2.13 (7H, m), 4.22 (2H, s), 4.80 (2H, broad d), 6.54 (1H, m), 7.12 (2H, m), 7.50 (2H, d), 8.48 (2H, d), 9.20 (1H, broad s). Mass Spectrum (Electrospray LC/MS): Found 428 (MH⁺). C₂₃H₂₃F₂N₃O₃ requires 427. Ret. time 2.95 min.

### Description 6. Methyl amino{4-[(methyloxy)methyl]phenyl}acetate

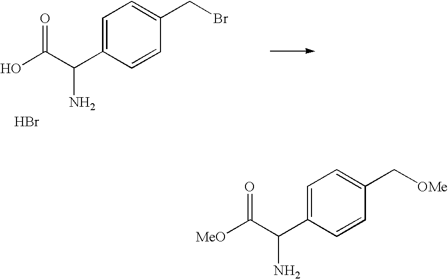

A stirred solution of amino[4-(bromomethyl)phenyl]acetic acid hydrobromide (Tetrahedron 1977, 33(20), 2715) (5.0 g, 0.015 mol) in methanol (120 ml) was heated under argon at reflux with stirring for 40 hours. Conversion of bromomethyl to methoxymethyl was complete and approx. 55:45 mixture of acid:methyl ester was present. The mixture was treated with conc. HCl acid (4 ml) and heated at reflux for 8 hours, followed by 16 hours at room temperature. The reaction mixture was concentrated under vacuum to approx. 15 ml, then the residue treated with sat. NaHCO₃ solution (80 ml) and extracted with ether (2×60 ml). The combined extract was dried (Na₂SO₄) and concentrated under vacuum to give the title compound a pale yellow oil (1.1 g). Extraction of the aqueous with dichloromethane (3×60 ml), combining the extracts and concentrating under vacuum afforded a further batch of title compound as a colourless oil (1.5 g). Mass Spectrum (Electrospray LC/MS): Found 210 (MH⁺) very weak. C₁₁H₁₅NO₃ requires 209. Ret. time 1.16 min.

¹H NMR δ (CDCl₃; 400 MHz): 2.03 (2H, br s), 3.38 (3H, s), 3.67 (3H, s), 4.44 (2H, s), 4.62 (1H, s), 7.30-7.40 (4H, m).

### Description 7. 2-Amino-2-{4-[(methyloxy)methyl]phenyl}acetamide

### Method A

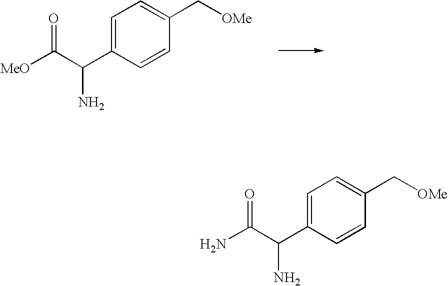

A mixture of methyl amino{4-[(methyloxy)methyl]phenyl}acetate (2.6 g, 0.012 mol, Description 6) and 0.88 ammonia solution (60 ml) was stirred well at room temperature for 3.5 days producing a colourless solution. This was extracted with dichloromethane (6×50 ml) and the combined extract dried (Na₂SO₄) and concentrated under vacuum to give the title compound as a white solid (1.34 g, 56%). Concentration of the aqueous under vacuum afforded a further batch of title compound as a white solid (0.86 g, 36%). Mass Spectrum (Electrospray LC/MS): Found 195 (MH⁺) very weak. C₁₀H₁₄N₂O₂ requires 194. Ret. time 0.63 min.

¹H NMR δ (CDCl₃; 400 MHz): 1.83 (2H, br s), 3.38 (3H, s), 4.44 (2H, s), 4.52 (1H, s), 5.91 (1H, br s), 6.88 (1H, br s), 7.33 (2H, d), 7.41 (2H, d).

### Method B

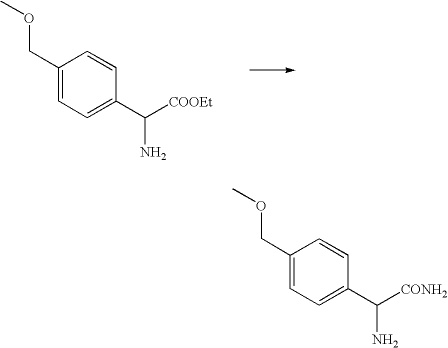

Ethyl amino{4-[(methyloxy)methyl]phenyl}acetate (37.7 g, 169 mmol, Description 16) was poured in a 500 mL pear flask. 28% ammonia solution (370 ml, 4787 mmol) was added and the reaction was allowed to stir overnight at room temperature. The mixture was evaporated under vacuum to dryness and then triturated in diethyl ether and filtered. A gummy solid was obtained, that was dissolved in methanol. The insoluble white solid was filtered and the methanol solution was evaporated in vacuum. The resulting solid was taken up in a mixture of diethyl ether/pentane 50 ml/250 ml and filtered. The collected solid was dried in vacuum until constant weight to get 25.9 g (77%) of title material as a solid. ¹H NMR (DMSO-d₆) δ: 2.3 (2H, broad s), 3.25 (3H, s), 4.3 (1H, s), 4.40 (2H, s), 7.0 (1H, broad s), 7.21 (2H, d), 7.35 (2H, d), 7.45 (1H, broad s).

### Description 8. 3-{4-[(Methyloxy)methyl]phenyl}-1,4-diazaspiro[4.5]decan-2-one

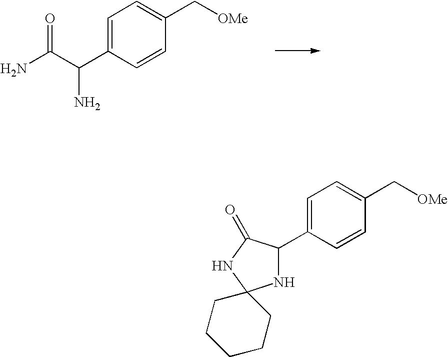

In a 4 L round-bottomed flask 2-amino-2-{4-[(methyloxy)methyl]phenyl}acetamide (30.5 g, 157 mmol, Description 7, Method B) was dissolved in ethanol 2.4 L to give a yellow solution. Cyclohexanone (15.41 g, 157 mmol) was added followed by 44 g of zeolite HY (product CBV400 from Zeolyst, Oosterhorn, Netherlands). The mixture was heated to reflux and allowed to stir at this temperature for 72 h, then it was cooled to room temperature, filtered and evaporated under vacuum to obtain 30 g of crude material that was triturated under magnetic stirring with 250 ml of a mixture of pentane/diethyl ether 95/5 to afford 27.5 g (61.3%) of title product as a white solid. ¹H NMR (CDCl₃) δ: 1.40-1.8 (10H, m), 2.15 (1H, broad s), 3.38 (3H, s), 4.48 (2H, s), 4.78 (1H, broad s), 6.58 (1H, broad s), 7.35 (2H, d), 7.48 (2H, d).

### Description 9. 3-{4-[(Methyloxy)methyl]phenyl}-1,4-diazaspiro[4.5]dec-3-en-2-one

### Method A

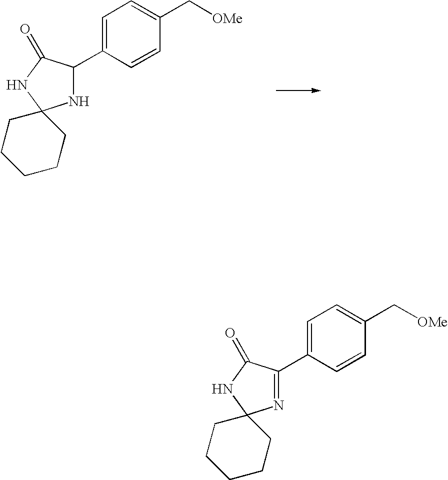

Reaction was carried out in flask covered with aluminium foil, protected from light. To a solution of 3-{4-[(methyloxy)methyl]phenyl}-1,4-diazaspiro[4.5]decan-2-one (15.4 g, 56.1 mmol, Description 8) in DCM (300 ml), a solution of N-bromosuccinimide (11 g, 61.7 mmol) in DCM (300 ml) was added dropwise in 20 min. The mixture was stirred at room temperature for 30 min. A solution of Na₂SO₃ (6 g in 300 ml of H₂O) was then added and the resulting mixture was stirred for 15 min. A sat sol of NaHCO₃ (400 ml) was added and the mixture diluted with DCM (400 ml). The organic phase was washed with HCl acid (2N, 2×200 ml), dried over Na₂SO₄ and concentrated in vacuum to give the title material (10.6 g, 69.3%) as white solid. ¹H NMR (CDCl₃) δ: 1.50-2.10 (10H, m), 3.43 (3H, s), 4.55 (2H, s), 7.45 (2H, d), 8.00 (1H, broad s), 8.41 (2H, d).

### Method B

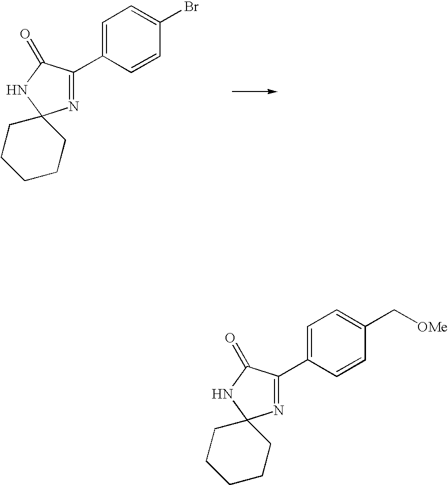

To potassium hydride (0.508 g, 3.80 mmol, 1.2 eq, 30% slurry in mineral oil) under argon at 0° C. was added a suspension of 3-(4-bromophenyl)-1,4-diazaspiro[4.5]dec-3-en-2-one (0.974 g, 3.17 mmol, 1.0 eq, Description 3) in anhydrous THF (15 ml) slowly over 10 min. The reaction mixture was allowed to warm to room temperature and stirred for a further 30 min. The yellow solution was cooled to −78° C. and then tert-butyllithium (2.2 ml, 3.80 mmol, 1.2 eq, 1.7M in pentane) was added dropwise over 5 min, giving a dark red/brown solution. After a further 20 min at −78° C. chloromethyl methyl ether (0.60 ml, 7.93 mmol, 2.5 eq) was added slowly. The resultant solution was stirred for 2 h at −78° C. then quenched with saturated NH₄Cl solution and allowed to warm to room temperature. The mixture was extracted twice with ethyl actetate, then the organic phase washed with water and brine and dried over MgSO₄. Concentration, followed by flash column chromatography over silica, eluting with 0-50% ethyl acetate in hexane gave the desired product as a white solid (312 mg, 36%).

### Description 10. 3-{4-[(Methyloxy)methyl]phenyl}-1,4-diazaspiro[4.4]nonan-2-one

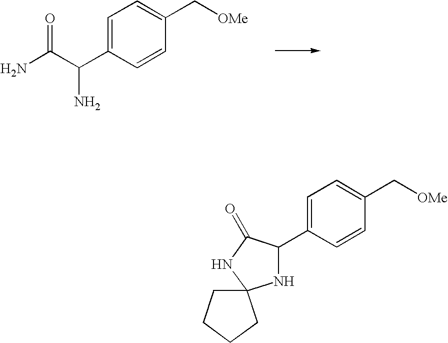

A stirred solution of 2-amino-2-{4-[(methyloxy)methyl]phenyl}acetamide (450 mg, 2.317 mmol, Description 7, Method A) in ethanol (20 ml) was treated with cyclopentanone (0.215 ml, 2.433 mmol) followed by Zeolite HY (product CBV400 from Zeolyst, Oosterhorn, Netherlands) (700 mg) and then heated at reflux for a total of 6 hours, plus overnight at room temperature. The mixture was allowed to cool, filtered through Kieselguhr and the filtrate concentrated under vacuum. The residue was crystallised from a mixture of dichloromethane (2 ml) and ether (8 ml) to give the title compound as a white solid (270 mg). Mass Spectrum (Electrospray LC/MS): Found 261 (MH⁺) weak. C₁₅H₂₀N₂O₂ requires 260. Ret. time 1.24 min.

¹H NMR δ (CDCl₃; 400 MHz): 1.60-1.85 (6H, m), 1.85-2.00 (2H, m), 2.25 (1H, br s), 3.73 (3H, s), 4.46 (2H, s), 4.65 (1H, s), 6.87 (1H, br s), 7.34 (2H, d), 7.46 (2H, d).

### Description 11. 3-{4-[(Methyloxy)methyl]phenyl}-1,4-diazaspiro[4.4]non-3-en-2-one

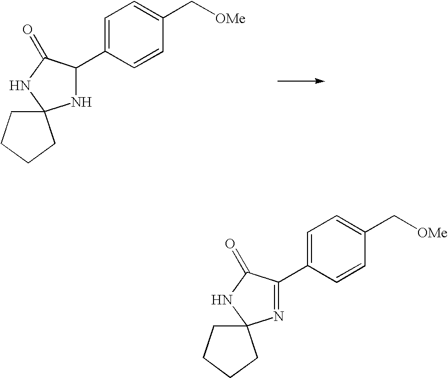

A stirred solution of 3-{4-[(methyloxy)methyl]phenyl}-1,4-diazaspiro[4.4]nonan-2-one (355 mg, 1.364 mmol, Description 10) in dichloromethane (20 ml) at room temperature under argon was treated with solid N-bromosuccinimide (243 mg, 1.364 mmol) and maintained at room temperature for 45 minutes. The orange solution was treated with saturated NaHCO₃ solution (15 ml) and stirred well. The orange colour was quickly lost. After 20 minutes the dichloromethane layer was separated, dried (Na₂SO₄) and concentrated under vacuum to leave a pale brown solid (302 mg), containing the title compound, as the major component. This was used directly in the next step. Mass Spectrum (Electrospray LC/MS): Found 259 (MH⁺). C₁₅H₁₈N₂O₂ requires 258. Ret. time 2.31 min.

¹H NMR δ (CDCl₃; 400 MHz): 1.90-2.20 (8H, m), 3.41 (3H, s), 4.53 (2H, s), 7.43 (2H, d), 8.37 (2H, d), 9.08 (1H, br s).

### Description 12. 2-Bromo-N-(3,5-difluorophenyl)acetamide

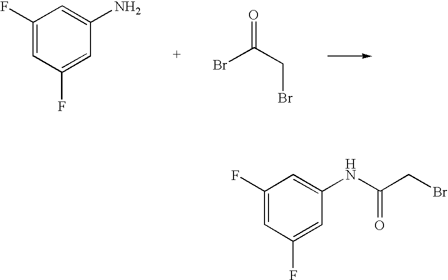

A mixture of 3,5-difluoroaniline (10 g; 77.45 mmol) and bromoacetyl bromide (6.73 ml; 77.45 mmol) in anhydrous dioxan (100 ml) was refluxed for 1.5 h, cooled to room temperature and diluted with water (400 ml) to afford a gum. The mother liquors were decanted and water added, followed by ethyl acetate. After stirring for 10 min the layers were separated and the organics dried and evaporated under reduced pressure. Recrystallisation from ethyl acetate-pentane afforded the title product as pale yellow crystals (6.5 g; 33%). ¹H NMR (CDCl₃) δ: 4.02 (2H, s), 6.60-6.65 (1H, m), 7.14-7.20 (2H, m), and 8.16 (1H, br s).

### Description 13. 2-Bromo-N-[3-(trifluoromethyl)phenyl]acetamide

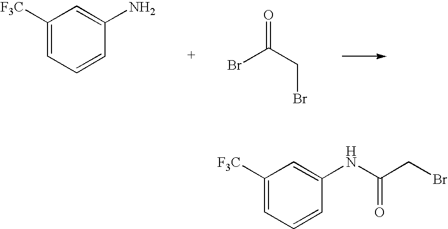

A stirred solution of 3-(trifluoromethyl)aniline (2.0 g, 0.012 mol) in dichloromethane (60 ml) at 10° C. under argon was treated dropwise over 5 minutes with bromoacetyl bromide (1.2 ml, 0.0137 mol). A white precipitate formed. This was allowed to warm to room temperature with good stirring over 1.5 hours, then treated with solid sodium hydrogen carbonate (1.65 g, 0.0196 mol) and stirred well for 40 minutes. The mixture was treated with water (100 ml), stirred well for 10 minutes, then the dichloromethane layer was isolated by passage through a phase separation cartridge and concentrated under vacuum to afford the title compound as a colourless oil (3.65 g, 100%). Mass Spectrum (Electrospray LC/MS): Found 282 (MH⁺). C₉H₇⁷⁹BrF₃NO requires 281. Ret. time 2.74 min. ¹H NMR δ (CDCl₃; 400 MHz): 4.05 (2H, s), 7.40-7.53 (2H, m), 7.76 (1H, d), 7.83 (1H, s), 8.24 (1H, br s).

### Description 14: (4-Bromophenyl)methyl methyl ether

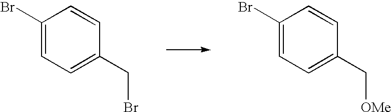

In a 2000 ml pear flask 1-bromo-4-(bromomethyl)benzene (142.2 g, 569 mmol) was treated with methanol (580 ml) to give a colourless suspension. Sodium methoxide (35.6 g, 626 mmol) was then added portionwise. The reaction was slightly exothermic and at the end of the addition a clear solution was observed and the internal temperature rose to 40° C. After the exothermic phase the reaction was refluxed for 2 h then the mixture was cooled to room temperature, concentrated under vacuum and finally partitioned between 2500 ml of ethyl acetate and 1000 ml of water. The organic phase was washed with 2×500 ml of water, then 500 ml of brine and finally dried over anhydrous sodium sulphate. The organic was evaporated under vacuum to obtain the title product as a colourless oil (110.6 g; 96%). ¹H NMR (CDCl₃) δ: 3.40 (3H, s), 4.41 (2H, s), 7.20 (2H, d), 7.50 (2H, d).

### Description 15: Ethyl [(diphenylmethylidene)amino] {4-[(methyloxy)methyl]phenyl}acetate

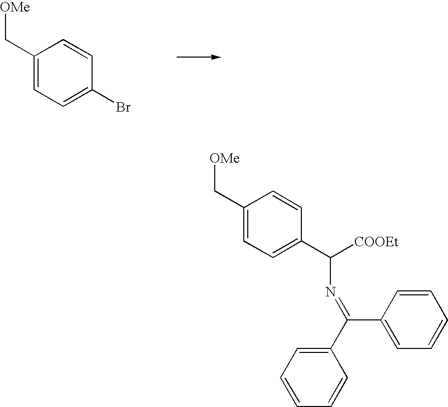

In a 2000 ml round-bottomed flask (4-bromophenyl)methyl methyl ether (30 g, 149 mmol, Description 14) was dissolved in toluene (500 ml) to give a yellow solution, followed by ethyl N-(diphenylmethylidene)glycinate (34 g, 127 mmol) and potassium phosphate (95 g, 448 mmol). Bis(tri-t-butylphosphine)palladium (0) (0.83 g, 1.624 mmol) was then added and the mixture was heated to 100° C. for 18 h.

According to the described procedure another 2 batches of ethyl N-(diphenylmethylidene)glycinate were processed as follows:

In a 2000 ml round-bottomed flask was (4-bromophenyl)methyl methyl ether (50 g, 249 mmol, Description 14) was dissolved in toluene (600 ml) to give a yellow solution, followed by ethyl N-(diphenylmethylidene)glycinate (63.2 g, 236 mmol) and potassium phosphate (158 g, 746 mmol). Bis(tri-t-butylphosphine)palladium (0) (1.271 g, 2.487 mmol) was added and the mixture was heated to 100° C. After 20 h another 5 g of (4-bromophenyl)methyl methyl ether and 500 mg of catalyst were added and the mixture was allowed to stir at 100° C. for further 3 h.

In a 500 ml round-bottomed flask was (4-bromophenyl)methyl methyl ether (10 g, 49.7 Mmol, Description 14) was dissolved in toluene (120 ml) to give a yellow solution, followed by ethyl N-(diphenylmethylidene)glycinate (14.63 g, 54.7 mmol) and potassium phosphate (31.7 g, 149 mmol) were added. Bis(tri-t-butylphosphine)palladium (0) (0.254 g, 0.497 mmol) was added and the mixture was heated to 100° C. After 5 h another 2 g of (4-bromophenyl)methyl methyl ether dissolved in 10 ml of toluene was added and the mixture was allowed to stir at 100° C. overnight. The day after another 2.5 g of (4-bromophenyl)methyl methyl ether dissolved in 10 ml of toluene was added and the mixture stirred at 100° C. further 3 h.

The 3 slurries were cooled down to room temperature, mixed homogeneously to give one single batch of crude material and worked up together. The crude was filtered, the organic phase was collected and the solid (inorganic salts) were dissolved in water and extracted with 3×250 ml of ethyl acetate. The combined organic phases were evaporated under vacuum until dryness and the residue oil (about 200 g) was taken up with the minimum amount of ethyl acetate (100 ml). A strong precipitation was observed so the solid was filtered and washed with pentane (2×80 ml) and finally dried under vacuum to afford 84.5 g of title product. The mother liquor was concentrated and purified by flash chromatography over a silica gel pad (SiO₂ 1200 g) eluting with cyclohexane/EtOAc from 9/1 to 8/2. The collected fractions were evaporated under vacuum to afford 120 g of oil. To this oil about 180 ml of a mixture pentane/Et₂O 90/10 was added. Slow formation of a precipitate was observed. The suspension was kept under vigorous stirring for 45 minutes then filtered. The collected solid was washed with 2×60 ml of pentane and finally dried in vacuum to obtain further 24.9 g of title product.

Overall amount of title material recovered was 109.4 g, yield=67.5% calculated wrt the limiting reagent, ethyl N-(diphenylmethylidene)glycinate. ¹H NMR (CDCl₃) δ: 1.22 (3H, t), 3.40 (3H, s), 4.10 (2H, q), 4.46 (2H, s), 5.15 (1H, s), 7.10 (2H, m), 7.20-7.50 (10H, m), 7.75 (2H, m).

### Description 16: Ethyl amino{4-[(methyloxy)methyl]phenyl}acetate

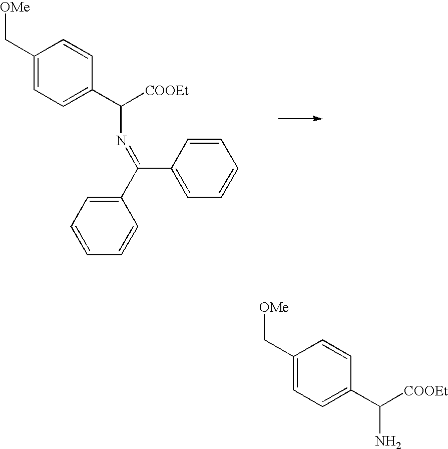

In a 2000 ml round-bottomed flask ethyl [(diphenylmethylidene)amino] {4-[(methyloxy)methyl]phenyl}acetate (88 g, 227 mmol, Description 15) was dissolved in ethanol (800 ml) to give a yellow solution. 6M HCl acid (500 ml, 3 mol) was added and the resulting mixture was stirred for 2 h.

According to this procedure a second batch of 20 g of ethyl [(diphenylmethylidene)amino] {4-[(methyloxy)methyl]phenyl}acetate was processed and the two crude materials were worked up together. The mixture was neutralised by portionwise addition of solid potassium carbonate (final pH about 7-8). A strong precipitation was observed and to this suspension 1.5 L of ethyl acetate was added and the remaining salts were filtered. The two phases were separated; the salts were dissolved in water and the combined aqueous phases were extracted with 2×500 ml of ethyl acetate. The combined organics were washed with 500 ml of 5% NaHCO₃ solution. The product was extracted from the organic solution as the hydrochloride salt with 2×500 ml of 3N HCl acid. To the aqueous acidic phase 1 L of ethyl acetate was added and solid potassium carbonate was carefully added portionwise under vigorous stirring to pH=8. The organic phase was dried over sodium sulphate and evaporated under vacuum to give 40 g of a crude material that was triturated with a mixture of pentane/diethyl ether 95/5 (250 ml) to obtain 37.7 g (y=63%) of desired product as a white solid.

¹H NMR (CDCl₃) δ: 1.25 (3H, t), 1.90 (2H, broad s), 3.40 (3H, s), 4.18 (2H, m), 4.48 (2H, s), 4.62 (1H, s), 7.32 (2H, d), 7.40 (2H, d).

### Description 17: 2-Chloro-N-(3,5-difluorophenyl)acetamide

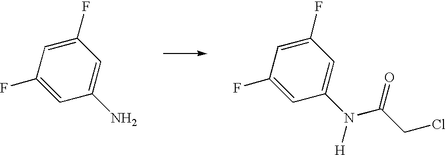

To a solution of 3,5-difluoroaniline (35 g) in dry toluene (350 ml) triethylamine (45.3 ml) was added. The solution was cooled to 0° C., then chloroacetyl chloride (22.80 ml, 285 mmol) was added dropwise over 45 min (a precipitate was formed). The mixture was stirred at room temperature for 1 hr. Water (300 ml) was added followed by ethyl acetate (300 ml), the phases were separated and the aqueous layer back extracted with ethyl acetate (2×300 ml). The combined organic phases were washed with water (200 ml), dried over Na₂SO₄, filtered and concentrated under vacuum to give 56 g of a brown solid that was triturated with pentane/Et₂O. Filtration afforded the title product (50.5 g, 91%) as light brown solid. ¹H NMR (CDCl₃) δ: 4.2 (2H, s), 6.65 (1H, m), 7.18 (2H, m), 8.3 (1H, bs).

### Description 18. 4-(3-Oxo-1,4-diazaspiro[4.4]non-1-en-2-yl)benzaldehyde

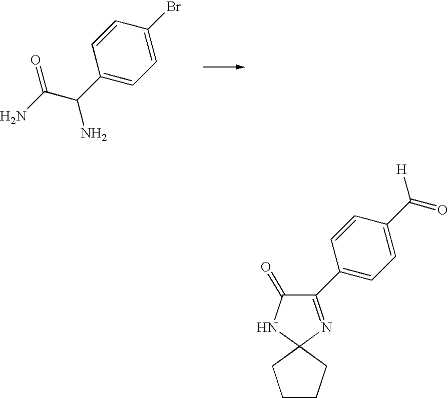

The title compound was prepared from 2-amino-2-(4-bromophenyl)acetamide (D1) and cyclopentanone using a similar conditions to those described in Description 1, followed by analogous procedures to those in Descriptions 2-4.

¹H NMR δ (CDCl₃; 400 MHz): 1.90-2.04 (4H, m), 2.04-2.20 (4H, m), 7.67 (1H, s), 7.98 (2H, dd), 8.58 (2H, dd), 10.10 (1H, s).

### EXAMPLE 1

### N-(3,5-Difluorophenyl)-2-[3-(4-formylphenyl)-2-oxo-1,4-diazaspiro[4.5]dec-3-en-1-yl]acetamide

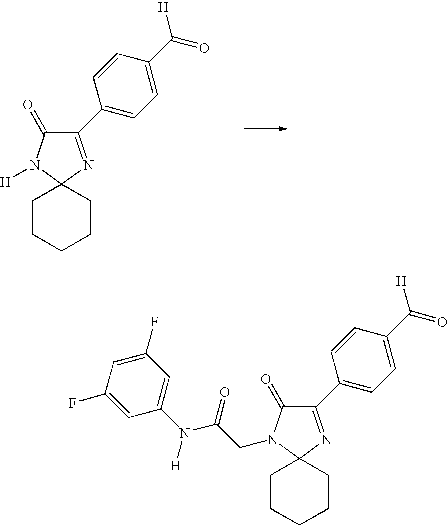

4-(3-Oxo-1,4-diazaspiro[4.5]dec-1-en-2-yl)benzaldehyde (0.762 g; 2.98 mmol, Description 4) and 2-bromo-N-(3,5-difluorophenyl)acetamide (1.118 g; 1.5 eq; 4.47 mmol, Description 12) were dissolved in dry DMF (25 ml) and stirred at room temperature under an atmosphere of argon for 16 hours in the presence of anhydrous potassium carbonate (815 mg; 2 eq; 5.96 mmol). The reaction mixture was then partitioned between ethyl acetate and saturated sodium hydrogen carbonate solution. The organic solution was dried (MgSO₄) and evaporated at reduced pressure. The residue was chromatographed over silica gel (50 g). Elution with a gradient of 0 to 50% ethyl acetate in pentane provided the title compound as a white foam (271 mg). ¹H NMR (CDCl₃; 400 MHz) δ: 1.40 (3H, broad d), 1.80-2.15 (7H, m), 4.28 (2H, s), 6.57 (1H, m), 7.10 (2H, m), 8.01 (2H, d), 8.66 (2H, d), 9.10 (1H, broad s), 10.12 (1H, s). Mass Spectrum (Electrospray LC/MS): Found 426 (MH⁺). C₂₃H₂₁F₂N₃O₃ requires 425. Ret. time 3.31 min.

### EXAMPLE 2

### N-(3,5-Difluorophenyl)-2-(3-{4-[(methyloxy)methyl]phenyl}-2-oxo-1,4-diazaspiro[4.5]dec-3-en-1-yl)acetamide

### METHOD A

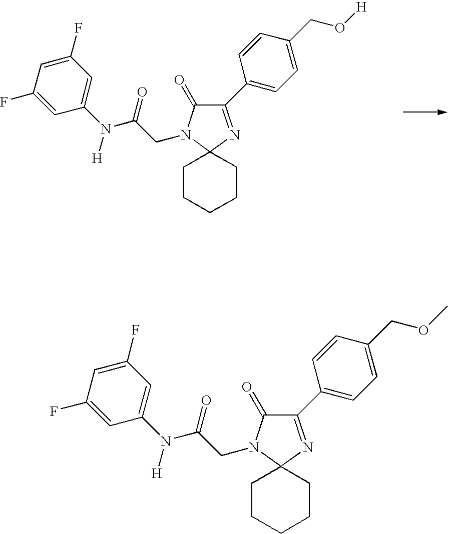

N-(3,5-Difluorophenyl)-2-{3-[4-(hydroxymethyl)phenyl]-2-oxo-1,4-diazaspiro[4.5]dec-3-en-1-yl}acetamide (200 mg: 0.469 mmol, Description 5) was dissolved in DCM (10 ml) and stirred at room temperature for 2 h under a atmosphere of argon with mesyl chloride (64.5 mg, 43.5 ul, 1.2 eq) and triethylamine (78 ul; 0.561 mmol). The reaction solution was then partitioned between DCM and water. The organic solution was separated, dried (MgSO₄) and evaporated at reduced pressure to yield the mesylate as a foam. Mass Spectrum (Electrospray LC/MS): Found 506 (MH⁺). C₂₄H₂₅F₂N₃SO₅ requires 505. Ret. time 3.22 min. The above product was dissolved in methanol (10 ml) and stirred at room temperature under an atmosphere of argon with sodium methoxide (51 mg; 2 eq; 0.944 mmol) for 16 h. The solution was then evaporated and partitioned between DCM and water. The organic solution was dried (MgSO₄), and evaporated at reduced pressure. The residue was purified by MDAP chromatography to yield the title compound as a colourless gum (97 mg, 47%). ¹H NMR (CDCl₃; 400 MHz) δ: 1.30-1.45 (3H, broad d), 1.79-2.11 (7H, m), 3.42 (3H, s), 4.22 (2H, s), 4.57 (2H, s), 6.54 (1H, m), 7.12 (2H, m), 7.48 (2H, d), 8.43 (2H, d), 9.21 (1H, broad s). Mass Spectrum (Electrospray LC/MS): Found 442 (MH⁺). C₂₄H₂₅F₂N₃O₃ requires 441. Ret. time 3.39 min.

### Method B

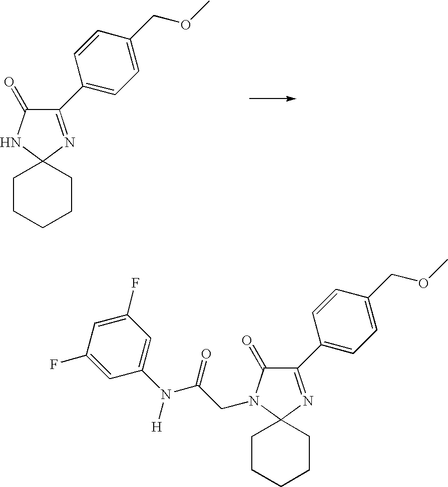

In a 500 mL pear-shaped flask 3-{4-[(methyloxy)methyl]phenyl}-1,4-diazaspiro[4.5]dec-3-en-2-one (17.2 g, 63.2 mmol, Description 9, Method B) was dissolved in N,N-dimethylformamide (DMF) (300 ml) to give a colourless solution. Potassium tert-butoxide (7.83 g, 66.3 mmol) was added and the solution turned yellow. After 30 minutes a catalytic amount of sodium iodide was added and then 2-chloro-N-(3,5-difluorophenyl)acetamide (12.98 g, 63.2 mmol, Description 17) dissolved in 100 ml of DMF was added dropwise. The reaction was allowed to stir at room temperature overnight. A further 2.1 g of 2-chloro-N-(3,5-difluorophenyl)acetamide (dissolved in 10 ml of DMF) was added and the mixture vigorously stirred at room temperature for 7 h. Another 783 mg of potassium tert-butoxide was then added and the mixture was allowed to stir on at room temperature overnight. The reaction mixture was partitioned between diethyl ether (1 L) and water (2 L); the aqueous was extracted with 2×200 ml of diethyl ether and the combined organic phase was washed with water (300 ml), brine (300 ml) and finally dried over anhydrous sodium sulphate and evaporated in vacuum to give 28.2 g of a crude oil that was purified by flash chromatography eluting with cyclohexane/ethyl acetate=7/3. Evaporation of the solvent afforded 18 g of desired product as a slightly yellow solid, which was triturated in diethyl ether (100 ml) to afford 15.9 g of desired material. 2 g of this material were re-crystallised from iPrOH (30 ml) to obtain 1.7 g of a white solid. This material was combined with the previously obtained solid, mixed homogeneously in DCM and evaporated in vacuum until dryness. The resulting solid was then recrystallised from 230 ml of iPrOH. After 5 h the crystalline solid was filtered and washed with cold iPrOH. The solid was dried in vacuum to give the title material (9.8 g, y=34.7%) a white crystalline solid. ¹H NMR (CDCl₃) δ: 1.20-1.45 (3H, broad d), 1.79-2.11 (7H, m), 3.42 (3H, s), 4.22 (2H, s), 4.57 (2H, s), 6.54 (1H, m), 7.12 (2H, m), 7.48 (2H, d), 8.43 (2H, d), 9.21 (1H, broad s).

The following examples were prepared by methods outlined in the reaction schemes. Example 3 was prepared by oxidation of Example 4 with meta-chloroperbenzoic acid. Example 4 was prepared from the product in Description 3 using procedures similar to those in Scheme 8 and 10. Example 5 was prepared from the product in Description 4 using procedures similar to those in Scheme 11.

| | | Mass spectrum | |
|---|---|---|---|
| | | (Electrospray LC/MS), | |
| | | APIP* | |
| Ex | Structure | Ret. time (min) | Name |
| 3 | 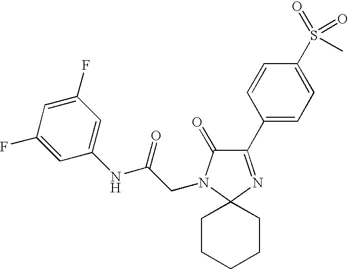 | Found 476 (MH⁺). C₂₃H₂₃F₂N₃O₄S requires 475. Ret. time 3.12 min. | N-(3,5-difluorophenyl)-2-{3- [4-(methylsulfonyl)phenyl]- 2-oxo-1,4- diazaspiro[4.5]dec-3-en-1- yl}acetamide |
| 4 | 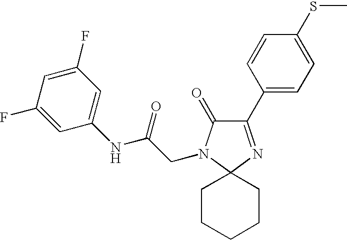 | Found 444 (MH⁺). C₂₃H₂₃F₂N₃O₂S requires .443. Ret. time 3.64 min. | N-(3,5-difluorophenyl)-2-{3- [4-(methylthio)phenyl]-2- oxo-1,4-diazaspiro[4.5]dec- 3-en-1-yl}acetamide |
| 5 | 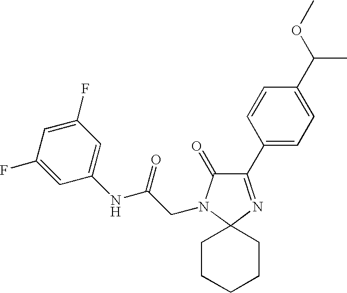 | Found 456 (MH⁺). C₂₅H₂₇F₂N₃O₃ requires 455. Ret. Time 3.56 min. | N-(3,5-difluorophenyl)-2-(3- {4-[1- (methyloxy)ethyl]phenyl}-2- oxo-1,4-diazaspiro[4.5]dec- 3-en-1-yl)acetamide |

### EXAMPLE 6

### 3-{4-[(Methyloxy)methyl]phenyl}-1-{N-[3-(trifluoromethyl)phenyl]glycyl}-1,4-diazaspiro[4.5]dec-3-en-2-one

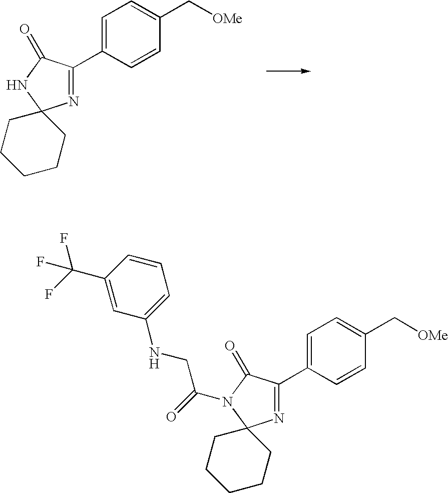

A stirred solution of 3-{4-[(methyloxy)methyl]phenyl}-1,4-diazaspiro[4.5]dec-3-en-2-one (170 mg, 0.624 mmol, Description 9, Method A) in dry dimethylformamide (2.75 ml) at 0° C. under argon was treated portionwise with sodium hydride (30 mg of 60% oil dispersion) and maintained for 30 minutes, then a solution of 2-bromo-N-[3-(trifluoromethyl)phenyl]acetamide (200 mg, 0.709 mmol, Description 13) in dry dimethylformamide (2.75 ml) was added via syringe pump over a period of 1 hour to the solution at 0° C. The reaction mixture was allowed to warm to room temperature with stirring over a period of 2.5 hours, then treated with water (30 ml) and extracted with EtOAc (2×20 ml). The combined extract was washed with water (25 ml), dried (Na₂SO₄) and concentrated under vacuum to leave a yellow oil (327 mg), which was purified by chromatography on a Biotage 25+M column eluting with 0-15% EtOAc/dichloromethane. Fractions containing the required material were combined and concentrated under vacuum to give a colourless oil, which was dissolved in ether (10 ml) and the title compound crystallised slowly on standing overnight as a white solid (133 mg). Mass Spectrum (Electrospray LC/MS): Found 474 (MH⁺). C₂₅H₂₆F₃N₃O₃ requires 473. Ret. time 3.52 min.

¹H NMR δ (CDCl₃; 400 MHz): 1.30-1.45 (3H, m), 1.80-1.96 (3H, m), 1.96-2.13 (4H, m), 3.42 (3H, s), 4.26 (2H, s), 4.54 (2H, s), 7.35 (1H, m), 7.40 (1H, m), 7.47 (2H, d), 7.65 (1H, d), 7.82 (1H, s), 8.44 (2H, d), 9.15 (1H, s).

### EXAMPLE 7

### 3-{4-[(Methyloxy)methyl]phenyl}-1-{N-[3-(trifluoromethyl)phenyl]glycyl}-1,4-diazaspiro[4.4]non-3-en-2-one

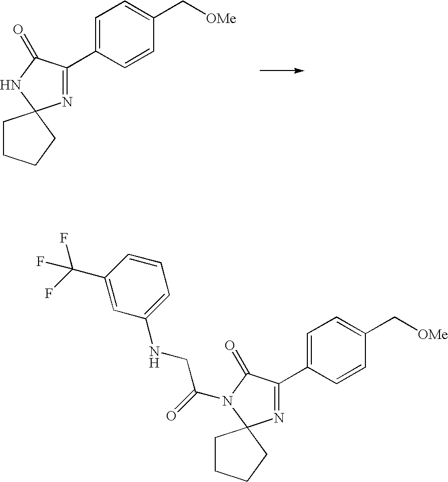

A stirred solution of 3-{4-[(methyloxy)methyl]phenyl}-1,4-diazaspiro[4.4]non-3-en-2-one (141 mg, 0.546 mmol, Description 11) in dry dimethylformamide (2.5 ml) at 0° C. under argon was treated with sodium hydride (26.2 mg, 0.655 mmol) and stirred well for 20 minutes, then a solution of 2-bromo-N-[3-(trifluoromethyl)phenyl]acetamide (185 mg, 0.655 mmol, Description 13) in dry dimethylformamide (2.5 ml) was added by syringe pump over 50 minutes. The reaction mixture was allowed to warm to room temperature over 1 hour, then treated with water (25 ml) and extracted with EtOAc (2×20 ml). The combined extract was washed with water (2×25 ml), dried (Na₂SO₄) and concentrated under vacuum to leave a pale brown solid (290 mg). This was purified by chromatography on a Biotage 25+M column eluting with 0-20% EtOAc/dichloromethane. Collected fractions containing required material were combined and concentrated under vacuum to afford the title compound as a white solid (140 mg, 50.2%). Mass Spectrum (Electrospray LC/MS): Found 460 (MH⁺). C₂₄H₂₄F₃N₃O₃ requires 459. Ret. time 3.28 min.

¹H NMR δ (CDCl₃; 400 MHz): 1.83-1.94 (2H, m), 1.97-2.22 (6H, m), 3.42 (3H, s), 4.27 (2H, s), 4.54 (2H, s), 7.34 (1H, m), 7.40 (1H, m), 7.47 (2H, d), 7.64 (1H, d), 7.84 (1H, s), 8.41 (2H, d), 9.18 (1H, s).

### EXAMPLE 8

### 1-[N-(3,5-Difluorophenyl)glycyl]-3-{4-[(methyloxy)methyl]phenyl}-1,4-diazaspiro[4.4]non-3-en-2-one

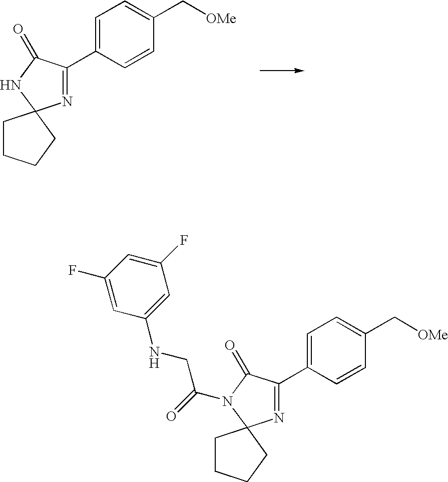

A stirred solution of 3-{4-[(methyloxy)methyl]phenyl}-1,4-diazaspiro[4.4]non-3-en-2-one (148 mg, 0.573 mmol, Description 11) in dry dimethylformamide (2.5 ml) at 0° C. under argon was treated with sodium hydride (27.5 mg, 0.688 mmol) and stirred for 25 minutes, then a solution of 2-bromo-N-(3,5-difluorophenyl)acetamide (172 mg, 0.688 mmol, Description 12) in dry dimethylformamide (2.5 ml) was added by syringe pump over 45 minutes. The reaction mixture was then allowed to warm to room temperature over 2 hours, then treated with water (30 ml) and extracted with EtOAc (2×20 ml). The combined extract was washed with water (40 ml), dried (Na₂SO₄) and concentrated under vacuum to leave a brown solid (244 mg). The residue was purified by chromatography on a Biotage 25+M column eluting with 0-20% EtOAc/dichloromethane. Collected fractions containing the required product were combined and concentrated under vacuum to leave a colourless oil (128 mg), which was crystallised from ether (7 ml) to afford the title compound as a crystalline white solid (90 mg). Mass Spectrum (Electrospray LC/MS): Found 428 (MH⁺). C₂₃H₂₃F₂N₃O₃ requires 427. Ret. time 3.17 min.

^{1P}H NMR δ (CDCl₃; 400 MHz): 1.80-1.93 (2H, m), 1.95-2.22 (6H, m), 3.42 (3H, s), 4.24 (2H, s), 4.54 (2H, s), 6.50-6.60 (1H, m), 7.08-7.16 (2H, m), 7.46 (2H, d), 8.40 (2H, d), 9.22 (1H, s).

### EXAMPLE 9

### N-(3,5-Difluorophenyl)-2-(3-{-4-[1-(methyloxy)ethyl]phenyl}-2-oxo-1,4-diazaspiro[4.4]non-3-en-1-yl)acetamide

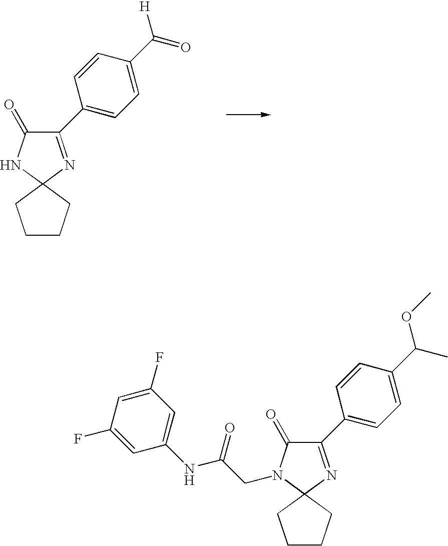

The title compound was prepared from 4-(3-oxo-1,4-diazaspiro[4.4]non-1-en-2-yl)benzaldehyde (D18) using procedures similar to those in Example 1, followed by a similar procedures to those in Scheme 11. Mass Spectrum (Electrospray LC/MS): Found 442 (MH⁺) C₂₄H₂₄F₂N₃O₃ requires 441. Ret. time 3.40 min.

¹H NMR δ (CDC₃; 400 MHz): 1.45 (3H, d), 1.80-1.92 (2H, m), 1.96-2.22 (6H, m), 3.25 (3H, s), 4.24 (2H, s), 4.37 (1H, q), 6.54 (1H, m), 7.10-7.17 (2H, m), 7.44 (2H, d), 8.40 (2H, d), 9.21 (1H, s).

### EXAMPLE 10

### N-(3,5-Difluorophenyl)-2-(3-{4-[(dimethylamino)methyl]phenyl}-2-oxo-1,4-diazaspiro[4.5]dec-3-en-1-yl)acetamide

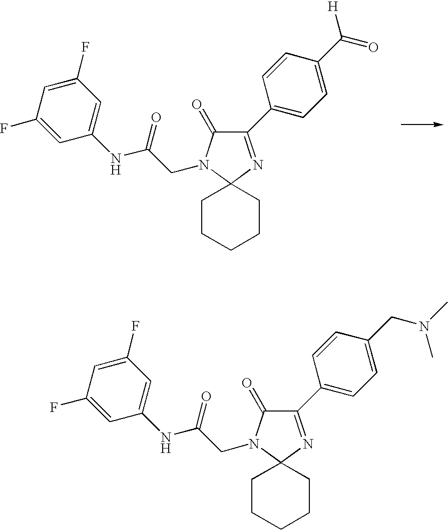

N-(3,5-difluorophenyl)-2-[3-(4-formylphenyl)-2-oxo-1,4-diazaspiro[4.5]dec-3-en-1-yl]acetamide (Example 1; 175 mg, 0.412 mmol) was dissolved in THF (20 ml) and dimethylamine solution in THF (2M, 0.412 ml, 0.824 mmol) was added, followed by titanium (IV) isopropoxide (0.824 mmol, 2.42 ul). The resulting solution was stirred at room temperature for 16 hours under argon. Sodium triacetoxyborohydride (0.618 mmol, 131 mg) was then added and stirring continued under argon at room temperature for a further 24 hours. Then it was quenched by the addition of saturated NaHCO₃ solution and EtOAc. The organic solution was washed with brine, dried (MgSO₄) and evaporated. The residue was purified by MDAP. Evaporation of the fractions gave a white solid which was dissolved in DCM and washed with NaHCO₃ solution. The DCM was passed through a phase separation cartridge and evaporated to yield the title product as a white foam (25 mg). Mass Spectrum (Electrospray LC/MS): Found 455 (MH⁺). C₂₅H₂₈F₂N₄O₂ requires 454. Ret. time 2.20 min.

¹H NMR δ (CDCl₃; 400 MHz): 1.30-1.45 (3H, m), 1.78-1.95 (3H, m), 1.95-2.12 (4H, m), 2.27 (6H, s), 3.51 (2H, s), 4.23 (2H, s), 6.50-6.57 (1H, m), 7.05-7.15 (2H, m), 7.45 (2H, d), 8.41 (2H, d), 9.21 (1H, s).

### EXAMPLE 11

### 4-(4-{2-[(3,5-Difluorophenyl)amino]-2-oxoethyl}-3-oxo-1,4-diazaspiro[4.5]dec-1-en-2-yl)-N,N-dimethylbenzamide

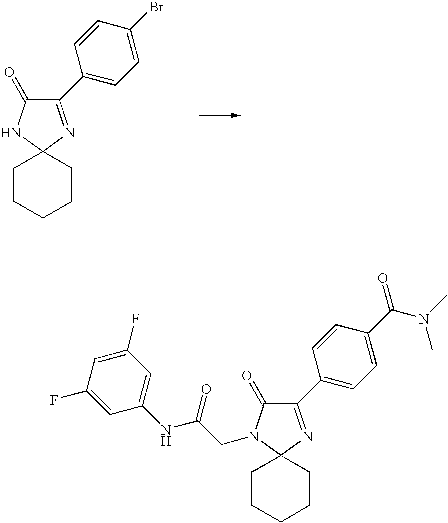

The title compound was prepared from 3-(4-bromophenyl)-1,4-diazaspiro[4.5]dec-3-en-2-one (D3) and N,N-dimethylcarbamoyl chloride using a analogous method to that in Description 4 followed by similar procedures to those described in Scheme 10. Mass Spectrum (Electrospray LC/MS): Found 469 (MH⁺) C₂₅H₂₆F₂N₄O₃ requires 468. Ret. time 3.02 min.

¹H NMR δ (CDC₃; 400 MHz): 1.30-1.45 (3H, m), 1.80-2.10 (7H, m), 2.99 (3H, s), 3.15 (3H, s), 4.16 (2H, s), 6.50-6.60 (1H, m), 7.08-7.18 (2H, m), 7.54 (2H, d), 8.51 (2H, d), 9.20 (1H, s).

1.-21. (canceled)

22. A compound of formula (I) or a salt thereof: 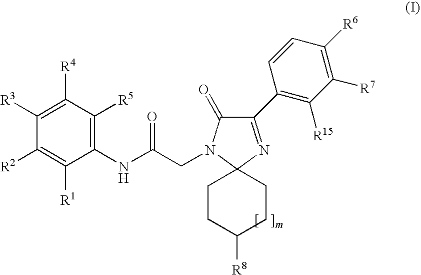
wherein:
R¹ is hydrogen, C₁₋₄alkyl, C₁₋₄alkoxy, halo, haloC₁₋₄ alkyl, haloC₁₋₄ alkoxy, C₁₋₄alkylthio, C₃₋₆cycloalkyl, C₃₋₆cycloalkylC₁₋₄ alkyl, C₁₋₄alkylsulfonyl, C₁₋₄alkoxyC₁₋₄alkyl, cyano, or C(O)NR^{a}R^{b} wherein R^{a} and R^{b} are independently H and C₁₋₄alkyl or R^{a} and R^{b} together with the nitrogen atom to which they are attached form a 4- to 7-membered ring;
R² is hydrogen, C₁₋₄alkyl, C₁₋₄alkoxy, halo, haloC₁₋₄ alkyl, haloC₁₋₄alkoxy, C₁₋₄alkylthio, C₃₋₆cycloalkyl, C₃₋₆cycloalkylC₁₋₄ alkyl, C₁₋₄alkylsulfonyl, C₁₋₄alkoxyC₁₋₄alkyl, cyano, or CONR^{c}R^{d} wherein R^{c} and R^{d} are independently selected from H and C₁₋₄alkyl or R^{c} and R^{d}, together with the nitrogen atom to which they are attached, form a 4- to 7-membered ring;
R³ is hydrogen, C₁₋₄alkyl, C₁₋₄alkoxy, halo, haloC₁₋₄ alkyl, haloC₁₋₄alkoxy, C₁₋₄alkylthio, C₃₋₆cycloalkyl, C₃₋₆cycloalkylC₁₋₄ alkyl, C₁₋₄alkylsulfonyl, C₁₋₄alkoxyC₁₋₄alkyl, cyano and CONR^{e}R^{f} wherein R^{e} and R^{f} are independently selected from H and C₁₋₄alkyl, or R^{e} and R^{f}, together with the nitrogen atom to which they are attached, form a 4- to 7-membered ring;
or R² and R³ together form the group —O—CH₂—O— or —O—CH₂—CH₂—O—;
R⁴ is hydrogen, C₁₋₄alkyl, C₁₋₄alkoxy, halo, haloC₁₋₄alkyl, haloC₁₋₄alkoxy, C₁₋₄alkylthio, C₃₋₆cycloalkyl, C₃₋₆cycloalkylC₁₋₄alkyl, C₁₋₄alkylsulfonyl, C₁₋₄alkoxyC₁₋₄alkyl, cyano, or CONR^{g}R^{h} wherein R^{g} and R^{h} are independently H or C₁₋₄alkyl, or R^{g} and R^{h} together with the nitrogen atom to which they are attached form a 4- to 7-membered ring;
R⁵ is hydrogen, chloro, fluoro, C₁₋₄alkyl or CF₃;
R⁶ is C₁₋₄alkoxyC₁₋₄alkyl, C₁₋₄alkylsulfonyl, C₁₋₄alkylthio, COR⁹ wherein R⁹ is hydrogen or C₁₋₄alkyl, CONRⁱR^{j} wherein Rⁱ and R^{j} are independently hydrogen or C₁₋₄alkyl or together with the nitrogen atom to which they are attached form a 4, 5 or 6-membered ring, or CHR^{k}NR^{l}R^{m} wherein R^{k} is hydrogen or C₁₋₄alkyl and R^{l} and R^{m} are independently hydrogen or C₁₋₄alkyl or R^{l} and R^{m}, together with the nitrogen atom to which they are attached, form a 4, 5 or 6-membered ring;
R¹⁵ is hydrogen or fluoro;
R⁷ is hydrogen, C₁₋₄alkyl, C₁₋₄alkoxy, haloC₁₋₄alkoxy, halo, cyano, C₁₋₄alkoxyC₁₋₄alkoxy and C₁₋₄alkoxyC₁₋₄alkyl;
R⁸ is hydrogen or methyl; and
m is 0, 1 or 2.

23. A compound as claimed in claim 22 wherein R¹ is hydrogen.

24. A compound as claimed in claim 22 wherein R² is halo.

25. A compound as claimed in claim 22 wherein R³ is hydrogen.

26. A compound as claimed in claim 22 wherein R⁴ is halo or hydrogen.

27. A compound as claimed in claim 22 wherein R⁵ is hydrogen.

28. A compound as claimed in claim 22 wherein R⁶ is C₁₋₂alkoxyC₁₋₂alkyl, C₁₋₂alkylsulfonyl, C₁₋₂alkylthio, COR⁹ wherein R⁹ is hydrogen or C₁₋₂alkyl, CONRⁱR^{j} wherein Rⁱ and R^{j} are independently hydrogen or C₁₋₂alkyl or together with the nitrogen atom to which they are attached form a 4, 5 or 6-membered ring, or CHR^{k}NR^{l}R^{m} wherein R^{k} is hydrogen or C₁₋₄alkyl and R^{l} and R^{m} are independently hydrogen or C₁₋₂alkyl or R^{l} and R^{m} together with the nitrogen atom to which they are attached form a 4, 5 or 6-membered ring.

29. A compound as claimed in claim 22 wherein R¹⁵ is hydrogen.

30. A compound as claimed in claim 22 wherein R⁷ is hydrogen.

31. A compound as claimed in claim 22 wherein R⁸ is hydrogen.

32. A compound as claimed in claim 22 which is:
N-(3,5-difluorophenyl)-2-{3-[4-(methylsulfonyl)phenyl]-2-oxo-1,4-diazaspiro[4.5]dec-3-en-1-yl}acetamide
N-(3,5-difluorophenyl)-2-{3-[4-(methylthio)phenyl]-2-oxo-1,4-diazaspiro[4.5]dec-3-en-1-yl}acetamide
N-(3,5-difluorophenyl)-2-[3-(4-formylphenyl)-2-oxo-1,4-diazaspiro[4.5]dec-3-en-1-yl]acetamide
N-(3,5-difluorophenyl)-2-(3-{4-[(methyloxy)methyl]phenyl}-2-oxo-1,4-diazaspiro[4.5]dec-3-en-1-yl)acetamide
N-(3,5-difluorophenyl)-2-(3-{4-[1-(methyloxy)ethyl]phenyl}-2-oxo-1,4-diazaspiro[4.5]dec-3-en-1-yl)acetamide
3-{4-[(methyloxy)methyl]phenyl}-1-{N-[3-(trifluoromethyl)phenyl]glycyl}-1,4-diazaspiro[4.5]dec-3-en-2-one
3-{4-[(methyloxy)methyl]phenyl}-1-{N-[3-(trifluoromethyl)phenyl]glycyl}-1,4-diazaspiro[4.4]non-3-en-2-one
1-[N-(3,5-difluorophenyl)glycyl]-3-{4-[(methyloxy)methyl]phenyl}-1,4-diazaspiro[4.4]non-3-en-2-one
N-(3,5-difluorophenyl)-2-(3-{4-[1-(methyloxy)ethyl]phenyl}-2-oxo-1,4-diazaspiro[4.4]non-3-en-1-yl)acetamide
N-(3,5-difluorophenyl)-2-(3-{4-[(dimethylamino)methyl]phenyl}-2-oxo-1,4-diazaspiro[4.5]dec-3-en-1-yl)acetamide
4-(4-{2-[(3,5-difluorophenyl)amino]-2-oxo ethyl}-3-oxo-1,4-diazaspiro[4.5]dec-1-en-2-yl)-N,N-dimethylbenzamide
and salts thereof.

33. A compound of formula (II) or a salt thereof: 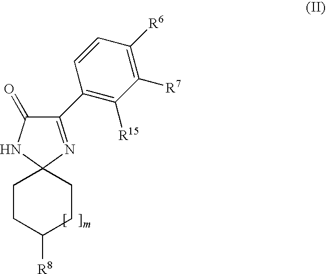
wherein:
R⁶ is C₁₋₄alkoxyC₁₋₄alkyl, C₁₋₄alkylsulfonyl, C₁₋₄alkylthio, COR⁹ wherein R⁹ is hydrogen or C₁₋₄alkyl), CONRⁱR^{j} wherein Rⁱ and R^{j} are independently hydrogen and C₁₋₄alkyl or together with the nitrogen atom to which they are attached form a 4, 5 or 6-membered ring, or CHR^{k}NR^{l}R^{m} wherein R^{k} is hydrogen or C₁₋₄alkyl and R^{l} and R^{m} are independently hydrogen or C₁₋₄alkyl or R^{l} and R^{m}, together with the nitrogen atom to which they are attached form a 4, 5 or 6-membered ring;
R¹⁵ is hydrogen or fluoro;
R⁷ is hydrogen, C₁₋₄alkyl, C₁₋₄alkoxy, haloC₁₋₄alkyl, haloC₁₋₄alkoxy, halo, cyano, C₁₋₄alkoxyC₁₋₄alkoxy or C₁₋₄alkoxyC₁₋₄alkyl;
R⁸ is hydrogen or methyl; and
m is 0, 1 or 2.

37. A method of treating schizophrenia, dementia or attention deficit disorder comprising administering to a patient in need thereof an effective amount of a compound of formula (I) or a pharmaceutically acceptable salt there of as defined in claim 22.

38. A method of treating schizophrenia, dementia or attention deficit disorder comprising administering to a patient in need thereof an effective amount of a compound of formula (II) or a pharmaceutically acceptable salt there of as defined in claim 33.

39. A pharmaceutical composition comprising a compound or formula (I) or a pharmaceutically acceptable salt thereof as claimed in claim 22 and at least one pharmaceutically acceptable excipient.

40. A pharmaceutical composition comprising a compound or formula (I) or a pharmaceutically acceptable salt thereof as claimed in claim 33 and at least one pharmaceutically acceptable excipient.
